SEQUENCE LISTING (38 sequences)

SEQ ID NO: 1 - DNA, 14216 bp, Artificial Sequence
  Synthetic DNA construct- >4_2019-nCoV S1-Ecto(1-1220) w signal peptide GSAS_pVL1393
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatccatgctgctggtgaaccagtctcaccagggtttcaacaaggagcacacctccaagatggtcagcgctatcgtgctgtacgtcctgctggctgctgctgctcactccgctttcgccatgttcctgctgaccactaagcgcaccatgttcgtgttcctggtcctgctgcctctggtgtccagccaatgcgtcaacctcactactcgtactcaactgcctccagcctacacaaactcgttcactcgtggcgtctactaccctgataaagtcttccgttccagcgtgctgcactctactcaggacctgttcctgcctttcttctctaacgtcacctggttccacgccatccacgtgtccggcaccaacggaactaagcgtttcgacaaccctgtgctgcccttcaacgacggtgtctacttcgcttctaccgagaagtcaaacatcatcaggggttggatcttcggcaccactctggactccaagactcagagcctgctgatcgtcaacaacgccaccaacgtggtcatcaaggtgtgcgagttccagttctgcaacgaccccttcctgggcgtgtactaccacaagaacaacaagtcttggatggagtcagagttcagggtctactcttcagctaacaactgcactttcgagtacgtgagccagccattcctgatggacctggaaggcaagcagggcaacttcaagaacctgagagagttcgtgttcaagaacatcgacggttacttcaagatctacagcaagcacaccccaatcaacctggtgcgcgacctgcctcagggtttctctgctctggagccactggtcgacctgcctatcggcatcaacatcacccgcttccagactctgctggctctgcaccgttcttacctgactccaggcgactccagctctggatggaccgctggtgctgctgcttactacgtgggatacctgcagcctcgtaccttcctgctgaagtacaacgaaaacggtaccatcactgacgccgtggactgcgctctggacccactgagcgaaaccaagtgcactctgaagtctttcaccgtcgagaagggaatctaccagactagcaacttcagggtgcagcctaccgaatctatcgtcagattccctaacatcactaacctgtgccccttcggagaggtcttcaacgccaccaggttcgctagcgtgtacgcctggaacaggaagagaatcagcaactgcgtcgctgactactctgtgctgtacaacagcgcctctttctcaaccttcaagtgctacggagtgtcacccactaagctgaacgacctgtgcttcaccaacgtctacgccgactccttcgtgatcaggggtgacgaggtcagacagatcgctcctggacagactggcaagatcgccgactacaactacaagctgcccgacgacttcaccggttgcgtcatcgcttggaactccaacaacctggacagcaaagtgggtggcaactacaactacctgtaccgcctgttccgtaagagcaacctgaagcccttcgagagagacatctcaactgaaatctaccaggccggttccaccccatgcaacggcgtggagggattcaactgctacttccccctgcagtcttacggcttccagccaactaacggagtcggttaccagccttaccgcgtggtcgtgctgagcttcgaactgctgcacgctccagctactgtgtgcggaccaaagaagtctaccaacctggtcaagaacaagtgcgtgaacttcaacttcaacggactgaccggcactggagtgctgaccgagagcaacaagaagttcctgcctttccagcagttcggtcgcgacatcgctgacaccactgacgccgtccgtgacccccagaccctggaaatcctggacatcactccatgctctttcggaggtgtctcagtgatcactcccggcaccaacacttctaaccaggtcgctgtgctgtaccaggacgtgaactgcaccgaggtccctgtggccatccacgctgaccagctgacccccacttggagggtgtactccaccggtagcaacgtcttccagactagagctggctgcctgatcggagccgagcacgtcaacaactcatacgaatgcgacatccctatcggtgctggcatctgcgcctcctaccagacccagactaactcaccaggttccgctagctccgtggcttcccagagcatcatcgcttacaccatgagcctgggagctgaaaactctgtcgcctactctaacaactcaatcgccatccccaccaacttcactatctcagtgaccactgagatcctgccagtctccatgaccaagactagcgtggactgcactatgtacatctgcggcgactccaccgaatgcagcaacctgctgctgcagtacggctccttctgcacccagctgaaccgcgctctgactggaatcgccgtggagcaggacaagaacactcaggaagtcttcgctcaggtgaagcagatctacaagacccctcccatcaaggacttcggcggattcaacttctcccagatcctgcccgacccatccaagcccagcaagcgttctttcatcgaggacctgctgttcaacaaggtgaccctggccgacgctggtttcatcaagcagtacggtgactgcctgggcgacatcgccgctagggacctgatctgcgctcagaagttcaacggcctgaccgtcctgccacctctgctgactgacgaaatgatcgcccagtacacttcagccctgctggctggaaccatcacttccggttggaccttcggagctggtgccgctctgcagatcccattcgctatgcagatggcctaccgcttcaacggcatcggagtcacccagaacgtgctgtacgagaaccagaagctgatcgctaaccagttcaactcagccatcggcaagatccaggactcactgtcatccactgcctccgctctgggaaagctgcaggacgtcgtgaaccagaacgcccaggctctgaacaccctggtcaagcagctgagctctaacttcggcgctatctcatccgtgctgaacgacatcctgtccagactggacaaggtcgaggccgaagtgcagatcgaccgcctgatcactggacgtctgcagtcactgcagacctacgtgactcagcagctgatcagggccgctgaaatcagagcctccgctaacctggccgctaccaagatgtcagagtgcgtcctgggccagtccaagcgcgtggacttctgcggcaagggctaccacctgatgtctttcccccagtcagctccacacggagtcgtgttcctgcacgtcacctacgtgccagcccaggagaagaacttcaccactgcccctgctatctgccacgacggcaaggctcacttccccagagaaggcgtcttcgtgagcaacggaacccactggttcgtgactcagcgcaacttctacgagccacagatcatcaccactgacaacactttcgtctctggaaactgcgacgtcgtgatcggtatcgtcaacaacaccgtgtacgacccactgcagcctgaactggacagcttcaaggaggaactggacaagtacttcaagaaccacacttctccagacgtggacctgggagacatctctggtatcaacgcttcagtcgtgaacatccagaaggagatcgaccgtctgaacgaagtcgccaagaacctgaacgaatccctgatcgacctgcaggagctgggcaagtacgaacagtacatcaagggagagaacctgtacttccagggtggaggaggtggctccggaggtggaggaagcggtggaatggtgtctaagggagaggaactgttcaccggcgtcgtgcctatcctggtggagctggacggcgacgtcaacggacacaagttctctgtctcaggagagggtgaaggcgacgctacctacggcaagctgactctgaagttcatctgcaccactggcaagctgcctgtgccctggccaaccctggtcaccactctgacttacggtgtgcagtgcttctcacgctacccagaccacatgaagcagcacgacttcttcaagtccgccatgcctgagggatacgtccaggaacgcaccatcttcttcaaggacgacggtaactacaagactcgtgctgaggtcaagttcgaaggagacaccctggtgaacaggatcgagctgaagggtatcgacttcaaggaagacggtaacatcctgggccacaagctggagtacaactacaactcacacaacgtctacatcatggccgacaagcagaagaacggtatcaaggtgaacttcaagatccgtcacaacatcgaagacggctccgtgcagctggctgaccactaccagcagaacactcctatcggagacggtcctgtgctgctgcccgacaaccactacctgtcaacccagtccgctctgagcaaggaccccaacgagaagagggaccacatggtcctgctggagttcgtgaccgccgctggcatcactctgggaatggacgaactctacaaacatcatcatcaccaccaccaccaccaccaccaccactaagaattccggagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 2 - DNA, 11264 bp, Artificial Sequence
  Synthetic DNA construct- S-RBD(319-591)-TEV- linker-eGFP-12XHistag cytoplasmic expression, >1_S1-RBD(319- 591)_2019-nCov_pVL1393
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatccatgcgtgtccagccaacggagtccattgtcagattccccaacattaccaacctgtgcccattcggagaagttttcaacgctaccagattcgcttccgtctacgcctggaaccgcaagcgtatcagcaactgcgtcgctgactactctgtgctgtacaactccgccagcttctctaccttcaagtgctacggcgtgtcaccaaccaagctgaacgacctgtgcttcactaacgtctacgctgactccttcgtgatccgcggagacgaggtccgtcagatcgctccaggacagaccggaaagatcgccgactacaactacaagctgccagacgacttcactggttgcgtgatcgcctggaactccaacaacctggacagcaaggtcggtggcaactacaactacctgtacaggctgttcagaaagagcaacctgaagccattcgagagagacatctcaaccgaaatctaccaggctggttccactccttgcaacggtgtcgagggcttcaactgctacttccccctgcagtcttacggtttccagccaaccaacggcgtgggataccagccttaccgcgtggtcgtgctgagcttcgaactgctgcacgctccagctaccgtgtgcggtcctaagaagtctactaacctggtcaagaacaagtgcgtgaacttcaacttcaacggcctgaccggtactggcgtcctgaccgagagcaacaagaagttcctgcccttccagcagttcggcagggacatcgctgacaccactgacgctgtgcgcgaccctcagactctggagatcctggacatcaccccttgctccggagaaaacctgtacttccagggaggtggaggaggttctggtggaggtggctcaggtggtatggtgtccaagggagaggaactgttcaccggcgtcgtgcctatcctggtggaactggacggagacgtcaacggtcacaagttctcagtgtccggagagggtgaaggcgacgccacctacggaaagctgactctgaagttcatctgcaccactggcaagctgcctgtgccctggccaaccctggtcaccactctgacttacggtgtccagtgcttcagccgctaccctgaccacatgaagcagcacgacttcttcaagtctgctatgcccgagggttacgtgcaggaacgcactatcttcttcaaggacgacggcaactacaagacccgtgccgaggtgaagttcgaaggcgacactctggtcaacaggatcgagctgaagggaatcgacttcaaggaagacggcaacatcctgggacacaagctggagtacaactacaactcacacaacgtgtacatcatggctgacaagcagaagaacggaatcaaggtcaacttcaagatccgtcacaacatcgaagacggttccgtgcagctggccgaccactaccagcagaacacccctatcggagacggtcctgtgctgctgcccgacaaccactacctgagcactcagtctgctctgtcaaaggaccccaacgagaagagggaccacatggtcctgctggagttcgtgaccgctgccggcatcactctgggtatggacgaactctacaagcatcaccaccaccaccaccaccaccaccaccaccactaagaattccggagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 3 - DNA, 11375 bp, Artificial Sequence
  Synthetic DNA construct- S-RBD(319-591)-TEV- linker-eGFP-12XHistag secreted, >2_S1-RBD(319-591)_2019-nCoV  w signal peptide_pVL1393
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatccatgctgctggtgaaccagtctcaccagggtttcaacaaggagcacacctccaagatggtcagcgctatcgtgctgtacgtcctgctggctgctgctgctcactccgctttcgcccgtgtccagccaacggagtccattgtcagattccccaacattaccaacctgtgcccattcggagaagttttcaacgctaccagattcgcttccgtctacgcctggaaccgcaagcgtatcagcaactgcgtcgctgactactctgtgctgtacaactccgccagcttctctaccttcaagtgctacggcgtgtcaccaaccaagctgaacgacctgtgcttcactaacgtctacgctgactccttcgtgatccgcggagacgaggtccgtcagatcgctccaggacagaccggaaagatcgccgactacaactacaagctgccagacgacttcactggttgcgtgatcgcctggaactccaacaacctggacagcaaggtcggtggcaactacaactacctgtacaggctgttcagaaagagcaacctgaagccattcgagagagacatctcaaccgaaatctaccaggctggttccactccttgcaacggtgtcgagggcttcaactgctacttccccctgcagtcttacggtttccagccaaccaacggcgtgggataccagccttaccgcgtggtcgtgctgagcttcgaactgctgcacgctccagctaccgtgtgcggtcctaagaagtctactaacctggtcaagaacaagtgcgtgaacttcaacttcaacggcctgaccggtactggcgtcctgaccgagagcaacaagaagttcctgcccttccagcagttcggcagggacatcgctgacaccactgacgctgtgcgcgaccctcagactctggagatcctggacatcaccccttgctccggagaaaacctgtacttccagggaggtggaggaggttctggtggaggtggctcaggtggtatggtgtccaagggagaggaactgttcaccggcgtcgtgcctatcctggtggaactggacggagacgtcaacggtcacaagttctcagtgtccggagagggtgaaggcgacgccacctacggaaagctgactctgaagttcatctgcaccactggcaagctgcctgtgccctggccaaccctggtcaccactctgacttacggtgtccagtgcttcagccgctaccctgaccacatgaagcagcacgacttcttcaagtctgctatgcccgagggttacgtgcaggaacgcactatcttcttcaaggacgacggcaactacaagacccgtgccgaggtgaagttcgaaggcgacactctggtcaacaggatcgagctgaagggaatcgacttcaaggaagacggcaacatcctgggacacaagctggagtacaactacaactcacacaacgtgtacatcatggctgacaagcagaagaacggaatcaaggtcaacttcaagatccgtcacaacatcgaagacggttccgtgcagctggccgaccactaccagcagaacacccctatcggagacggtcctgtgctgctgcccgacaaccactacctgagcactcagtctgctctgtcaaaggaccccaacgagaagagggaccacatggtcctgctggagttcgtgaccgctgccggcatcactctgggtatggacgaactctacaagcatcaccaccaccaccaccaccaccaccaccaccactaagaattccggagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 4 - DNA, 7252 bp, Artificial Sequence
  Synthetic DNA construct- >2_MBP-S1-RBD(319- 591)_pET-28a(+)
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatggcccatcatcatcatcatcatcatcatcatcacaagattgaagaaggtaaactggttatttggatcaacggcgacaagggctacaacggtctggcggaagtgggcaagaaatttgagaaagacaccggtattaaggttaccgtggaacacccggataaactggaggaaaagtttccgcaggttgcggcgaccggtgatggtccggatatcatcttctgggcgcacgaccgtttcggtggctatgcgcagagcggtctgctggcggaaatcaccccggacaaagcgtttcaagataagctgtacccgttcacctgggatgcggtgcgttataacggcaaactgatcgcgtatccgattgcggttgaggcgctgagcctgatttacaacaaagacctgctgccgaacccgccgaagacctgggaggaaatcccggcgctggataaggaactgaaggcgaaaggcaagagcgcgctgatgtttaacctgcaggagccgtattttacctggccgctgattgcggcggatggtggctatgcgttcaaatacgaaaacggcaaatacgacatcaaggatgttggtgtggacaacgcgggtgcgaaggcgggtctgacctttctggtggatctgattaaaaacaagcacatgaacgcggacaccgattatagcatcgcggaagcggcgttcaacaaaggcgaaaccgcgatgaccattaacggtccgtgggcgtggagcaacatcgataccagcaaggttaactacggcgttaccgtgctgccgacctttaaaggtcaaccgagcaagccgttcgttggcgtgctgagcgcgggtatcaacgcggcgagcccgaacaaagagctggcgaaggaatttctggagaactatctgctgaccgacgaaggcctggaggcggtgaacaaagataagccgctgggtgcggttgcgctgaaaagctacgaggaagagctggcgaaggacccgcgtattgcggcgacgatggagaacgcgcagaaaggcgagatcatgccgaacattccgcaaatgagcgcgttctggtacgcggttcgtaccgcggtgatcaacgcggcgagcggtcgtcagaccgtggacgaagcgctgaaagatgcgcaaaccggtggcggtagcggcggtggcagcggtggcggtagcggtgaaaacctgtattttcagggcggtcgtgtgcaaccgaccgagagcatcgttcgtttcccgaacattaccaacctgtgcccgtttggtgaggtgttcaacgcgacccgttttgcgagcgtttacgcgtggaaccgtaaacgtattagcaactgcgttgcggactatagcgtgctgtacaacagcgcgagcttcagcacctttaaatgctatggcgttagcccgaccaagctgaacgatctgtgctttaccaacgtttacgcggacagcttcgtgatccgtggtgatgaagttcgtcagattgcgccgggccaaaccggtaaaatcgcggactacaactataagctgccggacgatttcaccggttgcgtgatcgcgtggaacagcaacaacctggatagcaaggttggcggtaactacaactatctgtaccgtctgtttcgtaaaagcaacctgaagccgttcgaacgtgacatcagcaccgagatttatcaagcgggtagcaccccgtgcaacggtgtggaaggttttaactgctatttcccgctgcagagctacggttttcaaccgaccaacggcgttggttatcaaccgtaccgtgtggttgttctgagctttgagctgctgcatgcgccggcgaccgtgtgcggtccgaagaaaagcaccaacctggttaaaaacaagtgcgtgaacttcaactttaacggtctgaccggcaccggtgttctgaccgagagcaacaagaaattcctgccgtttcagcaattcggtcgtgacatcgcggataccaccgacgcggttcgtgacccgcagaccctggaaatcctggacattaccccgtgcagctaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 5 - DNA, 11699 bp, Artificial Sequence
  Synthetic DNA construct- N-TEV-linker-eGFP- 12XHistag cytoplasmic form, >10_nCoV19_N-tag(no Avi)_PVL1393
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatccatgtcggataacggaccgcaaaaccaacgcaacgcaccaaggattactttcggaggacccagtgatagcacaggctctaaccaaaacggcgagcgctcaggagctcgttccaagcagcgccgtccacagggactgcctaacaacaccgctagctggttcaccgccctgactcagcacggcaaggaagacctgaagttccctcgcggtcagggcgtgcccatcaacaccaactccagccccgacgaccagatcggttactacaggagagctaccaggcgtatcaggggtggcgacggcaagatgaaggacctgtctccaagatggtacttctactacctcggaactggtcctgaggctggtctgccatacggagccaacaaggacggcatcatctgggtggctaccgaaggagccctgaacactccaaaggaccacatcggcactcgtaaccctgctaacaacgctgccatcgtcctgcagctgccacagggaaccactctgcctaagggcttctacgctgagggaagccgcggaggttctcaggcctcttcacgttccagctctcgcagccgtaactcatccaggaactctacccctggaagctctaggggtacttcccccgctagaatggctggtaacggcggagacgctgctctggctctgctgctgctggacagactgaaccagctggagagcaagatgtctggcaagggacagcagcagcagggccagaccgtgactaagaagtcagctgccgaagcttccaagaagccaaggcagaagagaaccgctactaaggcctacaacgtcacccaggccttcggtaggagaggccctgagcagactcagggaaacttcggtgaccaggaactgatccgccagggtaccgactacaagcactggccccagatcgctcagttcgccccatcagcttccgccttcttcggaatgtcacgtatcggcatggaagtgaccccttccggaacttggctgacctacactggtgctatcaagctggacgacaaggaccccaacttcaaggaccaggtcatcctgctgaacaagcacatcgacgcctacaagaccttccctcccactgagcctaagaaggacaagaagaagaaggctgacgaaactcaggccctgccccagaggcagaagaagcagcagaccgtcactctgctgccagctgccgacctggatgacttctctaaacaactccagcaaagcatgtcctccgcagattcaacacaagcaggcgagaacctgtacttccaaggcggtggtggtggtagcggtggcggaggttctggcggaatggtgtccaagggagaggaactgttcaccggcgtggtccctatcctggtggagctggacggagacgtcaacggtcacaagttctccgtgagcggagagggcgaaggagacgctacctacggaaagctgactctgaagttcatctgcaccactggcaagctgcctgtgccctggccaaccctggtcaccactctgacttacggcgtccagtgcttctccaggtacccagaccacatgaagcagcacgacttcttcaagagcgctatgcctgagggttacgtgcaggaacgcactatcttcttcaaggacgacggcaactacaagacccgtgccgaggtgaagttcgaaggcgacactctggtcaacaggatcgagctgaagggaatcgacttcaaggaagacggcaacatcctgggacacaagctggagtacaactacaactcccacaacgtgtacatcatggctgacaagcagaagaacggaatcaaggtcaacttcaagatcagacacaacatcgaagacggttctgtgcagctggccgaccactaccagcagaacactcctatcggtgacggacctgtcctgctgcccgacaaccactacctgtctactcagtcagccctgtccaaggacccaaacgagaagcgcgaccacatggtgctgctggagttcgtcaccgctgccggtatcactctgggaatggacgaactctacaaacatcatcatcatcatcatcatcatcatcatcatcattaagaattccggagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 6 - DNA, 11813 bp, Artificial Sequence
  Synthetic DNA construct- N-TEV-linker-eGFP- 12XHistag secreted form, >15_signal peptide_nCoV19_N-tag(no Avi)_PVL1393
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatccatgctgctggtgaaccagtctcaccagggtttcaacaaggagcacacctccaagatggtcagcgctatcgtgctgtacgtcctgctggctgctgctgctcactccgctttcgccatgtcggataacggaccgcaaaaccaacgcaacgcaccaaggattactttcggaggacccagtgatagcacaggctctaaccaaaacggcgagcgctcaggagctcgttccaagcagcgccgtccacagggactgcctaacaacaccgctagctggttcaccgccctgactcagcacggcaaggaagacctgaagttccctcgcggtcagggcgtgcccatcaacaccaactccagccccgacgaccagatcggttactacaggagagctaccaggcgtatcaggggtggcgacggcaagatgaaggacctgtctccaagatggtacttctactacctcggaactggtcctgaggctggtctgccatacggagccaacaaggacggcatcatctgggtggctaccgaaggagccctgaacactccaaaggaccacatcggcactcgtaaccctgctaacaacgctgccatcgtcctgcagctgccacagggaaccactctgcctaagggcttctacgctgagggaagccgcggaggttctcaggcctcttcacgttccagctctcgcagccgtaactcatccaggaactctacccctggaagctctaggggtacttcccccgctagaatggctggtaacggcggagacgctgctctggctctgctgctgctggacagactgaaccagctggagagcaagatgtctggcaagggacagcagcagcagggccagaccgtgactaagaagtcagctgccgaagcttccaagaagccaaggcagaagagaaccgctactaaggcctacaacgtcacccaggccttcggtaggagaggccctgagcagactcagggaaacttcggtgaccaggaactgatccgccagggtaccgactacaagcactggccccagatcgctcagttcgccccatcagcttccgccttcttcggaatgtcacgtatcggcatggaagtgaccccttccggaacttggctgacctacactggtgctatcaagctggacgacaaggaccccaacttcaaggaccaggtcatcctgctgaacaagcacatcgacgcctacaagaccttccctcccactgagcctaagaaggacaagaagaagaaggctgacgaaactcaggccctgccccagaggcagaagaagcagcagaccgtcactctgctgccagctgccgacctggatgacttctctaaacaactccagcaaagcatgtcctccgcagattcaacacaagcaggcgagaacctgtacttccaaggcggtggtggtggtagcggtggcggaggttctggcggaatggtgtccaagggagaggaactgttcaccggcgtggtccctatcctggtggagctggacggagacgtcaacggtcacaagttctccgtgagcggagagggcgaaggagacgctacctacggaaagctgactctgaagttcatctgcaccactggcaagctgcctgtgccctggccaaccctggtcaccactctgacttacggcgtccagtgcttctccaggtacccagaccacatgaagcagcacgacttcttcaagagcgctatgcctgagggttacgtgcaggaacgcactatcttcttcaaggacgacggcaactacaagacccgtgccgaggtgaagttcgaaggcgacactctggtcaacaggatcgagctgaagggaatcgacttcaaggaagacggcaacatcctgggacacaagctggagtacaactacaactcccacaacgtgtacatcatggctgacaagcagaagaacggaatcaaggtcaacttcaagatcagacacaacatcgaagacggttctgtgcagctggccgaccactaccagcagaacactcctatcggtgacggacctgtcctgctgcccgacaaccactacctgtctactcagtcagccctgtccaaggacccaaacgagaagcgcgaccacatggtgctgctggagttcgtcaccgctgccggtatcactctgggaatggacgaactctacaaacatcatcatcatcatcatcatcatcatcatcatcattaagaattccggagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 7 - protein, 545 aa, Artificial Sequence
  Synthetic- S-RBD(319-591)-TEV-linker-eGFP- 12XHistag
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGENLYFQGGGGGSGGGGSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHHHHHHH

SEQ ID NO: 8 - protein, 554 aa, Artificial Sequence
  Synthetic- S-RBD(319-591)-TEV-linker-eGFP- 12XHistag
MFLLTTKRTMRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGENLYFQGGGGGSGGGGSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHHHHHHH

SEQ ID NO: 9 - protein, 1491 aa, Artificial Sequence
  Synthetic- S-Ecto(1-1220)GSAS-TEV-linker-eGFP- 12XHistag
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGENLYFQGGGGGSGGGGSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHHHHHHH

SEQ ID NO: 10 - protein, 1482 aa, Artificial Sequence
  Synthetic- S-Ecto(1-1220)GSAS-TEV-linker-eGFP- 12XHistag
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGENLYFQGGGGGSGGGGSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHHHHHHH

SEQ ID NO: 11 - protein, 690 aa, Artificial Sequence
  Synthetic- N-TEV-linker-eGFP-12XHistag
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGENLYFQGGGGGSGGGGSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHHHHHHH

SEQ ID NO: 12 - protein, 699 aa, Artificial Sequence
  Synthetic- N-TEV-linker-eGFP-12XHistag
MFLLTTKRTMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGENLYFQGGGGGSGGGGSGGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHHHHHHH

SEQ ID NO: 13 - protein, 671 aa, Artificial Sequence
  Synthetic- 10XHis-MBP-linker-TEV-S-RBD(319-591)
HHHHHHHHHHKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSGGGSGGGSGENLYFQGGMRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 14 - protein, 1273 aa, Artificial Sequence
  Synthetic- S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 419 aa, Artificial Sequence
  Synthetic- N protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 16 - protein, 222 aa, Artificial Sequence
  Synthetic- M protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 17 - protein, 75 aa, Artificial Sequence
  Synthetic- E protein
MYSFVSEETGTLIVNSVLLFLAFVVFLLVTLAILTALRLCAYCCNIVNVSLVKPSFYVYSRVKNLNSSRVPDLLV

SEQ ID NO: 18 - protein, 1611 aa, Artificial Sequence
  Synthetic- S-Ecto-GSAS-TEV-linker-MBP-12XHistag
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGENLYFQGGGGGSGGGGSGGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTHHHHHHHHHHHH

SEQ ID NO: 19 - protein, 1651 aa, Artificial Sequence
  Synthetic- S-Ecto-GSAS-TEV-linker-MBP-GGSK10- 12XHistag
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGENLYFQGGGGGSGGGGSGGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGSKGGSKGGSKGGSKGGSKGGSKGGSKGGSKGGSKGGSKHHHHHHHHHHHH

SEQ ID NO: 20 - protein, 674 aa, Artificial Sequence
  Synthetic- S-RBD(319-591)-TEV-linker-MBP- 12XHistag
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGSGENLYFQGGGGGSGGGGSGGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTHHHHHHHHHHHH

SEQ ID NO: 21 - protein, 714 aa, Artificial Sequence
  Synthetic- S-RBD(319-591)-TEV-linker-MBP- GGSK10-12XHistag
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGSGENLYFQGGGGGSGGGGSGGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGSKGGSKGGSKGGSKGGSKGGSKGGSKGGSKGGSKGGSKHHHHHHHHHHHH

SEQ ID NO: 22 - protein, 545 aa, Artificial Sequence; identical to SEQ ID NO: 7
  Synthetic- S-RBD(319-591)“-TEV-linker-eGFP- 12XHistag

SEQ ID NO: 23 - protein, 1491 aa, Artificial Sequence; identical to SEQ ID NO: 9
  Synthetic- S-Ecto(1-1220)GSAS-TEV-linker-eGFP- 12XHistag

SEQ ID NO: 24 - protein, 690 aa, Artificial Sequence; identical to SEQ ID NO: 11
  Synthetic- N-GFP; N-TEV-linker-eGFP-12XHistag

SEQ ID NO: 25 - protein, 699 aa, Artificial Sequence; identical to SEQ ID NO: 12
  Synthetic- N-TEV-linker-eGFP-12XHistag with secretion signal

SEQ ID NO: 26 - protein, 817 aa, Artificial Sequence
  Synthetic- MBP-N; 10XHistag-MBP-linker-TEV-N in pET28a
MAHHHHHHHHHHKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSGGGSGGGSGENLYFQGMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 27 - protein, 860 aa, Artificial Sequence
  Synthetic- 10XHistag-GGSK10-MBP-linker-TEV-N in pET28a
MAHHHHHHHHHHGGSKGGSKGGSKGGSKGGSKGGSKGGSKGGSKGGSKGGSKGGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSGGGSGGGSGENLYFQGMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 28 - protein, 671 aa, Artificial Sequence; identical to SEQ ID NO: 13
  Synthetic- 10X-His-MBP-linker-TEV-S-RBD(319- 591) in pET28a

SEQ ID NO: 29 - protein, 715 aa, Artificial Sequence
  Synthetic- 10X-His- GGSK10-MBP-linker-TEV-S- RBD(319-591) in pET28a
MAHHHHHHHHHHGGSKGGSKGGSKGGSKGGSKGGSKGGSKGGSKGGSKGGSKGGSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSGGGSGGGSGENLYFQGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 30 - protein, 671 aa, Artificial Sequence
  Synthetic- MBP-S1-RBD(319-591)Omicron 10XHis-MBP-linker-TEV-S-RBD(319-591)
HHHHHHHHHHKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSGGGSGGGSGENLYFQGGMRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVGSNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCS

SEQ ID NO: 31 - protein, 671 aa, Artificial Sequence
  Synthetic- MBP-S1-RBD(319-591)Delta 10XHis-MBP-linker-TEV-S-RBD(319-591)
HHHHHHHHHHKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGSGGGSGGGSGENLYFQGGMRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 32 - protein, 4 aa, Artificial Sequence
  Synthetic
GSAS

SEQ ID NO: 33 - protein, 4 aa, Artificial Sequence
  Synthetic
RRAR

SEQ ID NO: 34 - protein, 12 aa, Artificial Sequence
  Synthetic- linker
GGGGSGGGGSGG

SEQ ID NO: 35 - protein, 11 aa, Artificial Sequence
  Synthetic- protease site
GENLYFQGTEV

SEQ ID NO: 36 - protein, 9 aa, Artificial Sequence
  Synthetic- secretion signal
MFLLTTKRT

SEQ ID NO: 37 - protein, 8 aa, Artificial Sequence
  Synthetic- protease site
ENLYQTEV

SEQ ID NO: 38 - protein, 44 aa, Artificial Sequence
  Synthetic- GGSK10 linker
GGSKGGSKGGSKGGSKGGSKGGSKGGSKGGSKGGSKGGSKGGSK

(19) United States
(12) Patent Application Publication
(10) Pub. No.: US 2022/0339279 A1
(43) Pub. Date: Oct. 27, 2022

(54) RECOMBINANT PROTEINS, COMPOSITIONS, VECTORS, KITS, AND METHODS FOR IMMUNIZING AGAINST, AND TESTING FOR EXPOSURE TO, SEVERE ACUTE RESPIRATORY SYNDROME CORONAVIRUS 2

(71) Applicant: Board of Regents of the University of Nebraska, Lincoln, NE (US)

(72) Inventors: Kenneth Bayles, Omaha, NE (US); Gloria Borgstahl, Omaha, NE (US); Siddappa Byrareddy, Omaha, NE (US); Chittibabu Guda, Omaha, NE (US); St. Patrick Reid, Omaha, NE (US); Mara Jana Broadhurst, Omaha, NE (US); Andrew Schnaubelt, Papillion, NE (US)

(21) Appl. No.: 17/721,709

(22) Filed: Apr. 15, 2022

Related U.S. Application Data
(60) Provisional application No. 63/175,376, filed on Apr. 15, 2021.

(51) Int. Cl. A61K 39/215; C07K 14/165; A61P 31/14; G01N 33/569

(52) U.S. Cl. CPC A61K 39/215; C07K 14/165; A61P 31/14; G01N 33/56983; C07K 2319/50

(57) ABSTRACT

Disclosed are recombinant proteins, compositions, vectors, kits, data analyses, and methods for inducing an immune response against, or detecting exposure to, SARS-CoV-2. In particular, the compositions, vectors, kits, data analyses and methods may be utilized to immunize subjects against disease associated with SARS-CoV-2 infection or to protect subjects from SARS-CoV-2 infection. In some embodiments, the recombinant proteins are useful in the production of antibodies against SARS-CoV-2, and for the detection of exposure to SARS-CoV-2.

### CROSS-REFERENCE TO RELATED APPLICATIONS

This application claims the benefit of U.S. Provisional Application No. 63/175,376, filed Apr. 15, 2021, the contents of which is incorporated herein by reference in its entirety.

### SEQUENCE LISTING

A Sequence Listing accompanies this application and is submitted as an ASCII text file of the sequence listing named “170799 00016 ST25.txt” which is 271,557 bytes in size and was created on Apr. 15, 2022. The sequence listing is electronically submitted via EFS-Web with the application and is incorporated herein by reference in its entirety.

### FIELD

The present invention relates generally to the field of recombinant proteins, compositions, vectors, kits, data analyses, and methods for immunizing against coronaviruses and testing for exposure to coronaviruses. In particular, the invention relates to recombinant Severe Acute Respiratory Syndrome Coronavirus 2 (SARS-CoV-2) proteins, compositions, vectors, kits, data analyses and methods for immunizing subjects against infection by SARS-CoV-2, and testing for exposure to SARS-CoV-2.

### BACKGROUND

Coronavirus disease 2019 (COVID-2019) is caused by a novel coronavirus known as Severe Acute Respiratory Syndrome Coronavirus 2 (SARS-CoV-2) and was identified as a pandemic by the World Health Organization (WHO) on Mar. 11, 2020. As of Apr. 12, 2020, more than 1.8 million people were confirmed to have been infected and tested positive for COVID-19, with over 114,000 deaths worldwide. This virus was first identified in the respiratory tract of patients with pneumonia in Wuhan, Hubei China, in December 2019 which was then indicated as a newly identified β-coronavirus (nCoV). There is a need in the art for vaccines, antibodies, and related compositions to prevent, treat, and detect SARS-CoV-2 infection.

### SUMMARY

In a first aspect of the current disclosure, recombinant proteins are provided. In some embodiments, the recombinant proteins comprise: (i) a SARS-CoV-2 polypeptide sequence derived from the spike (“S”) protein or a variant thereof, and (ii) one or more heterologous polypeptide sequences selected from a purification tag, a detectable label, a flexible linker, a cleavage site to allow for tag removal after purification, and a secretion signal peptide. In some embodiments, the furin site “RRAR” in the polypeptide is genetically engineered so as not to be cleaved by furin, optionally wherein the furin site is engineered to “GSAS.” In some embodiments, the SARS-CoV-2 polypeptide sequence comprises a fragment of the S protein including amino acids 319-591, or a variant thereof. In some embodiments, the detectable label comprises green fluorescent protein (GFP) or enhanced green fluorescent protein (eGFP). In some embodiments, the recombinant proteins comprise one or more mutations selected from the group consisting of: F817P, A892P, A899P, A942P, K986P, and V987P, relative to SEQ ID NO: 14. In some embodiments, the flexible linker comprises GGGGSGGGGSGG (SEQ ID NO: 34). In some embodiments, the cleavage site to allow for tag removal after purification comprises a Tobacco Etch Virus nuclear-inclusion-a endopeptidase (TEV protease) recognition sequence: GENLYFQG (SEQ ID NO: 35). In some embodiments, the secretion signal peptide comprises MFLLTTKRT (SEQ ID NO: 36). In some embodiments, the recombinant proteins further comprise a foldon trimerization domain. In some embodiments, the recombinant proteins comprise a solubility enhancer peptide comprising maltose binding protein (MBP). In some embodiments, the maltose binding protein (MBP) comprises a GGSK₁₀ sequence (SEQ ID NO: 38) at its N terminus or C terminus. In some embodiments, the heterologous polypeptide sequence comprises (a) a purification tag comprising a HIS tag; (b) a detectable label comprising Green Fluorescent Protein or enhanced Green Fluorescent Protein; (c) a flexible linker comprising GGGGSGGGGSGG (SEQ ID NO: 34); (d) a cleavage site to allow for tag removal after purification comprising a Tobacco Etch Virus nuclear-inclusion-a endopeptidase (TEV protease) recognition sequence, GENLYFQG (SEQ ID NO: 35); (e) a secretion signal peptide comprising MFLLTTKRT (SEQ ID NO: 36); (f) a foldon trimerization domain; wherein the “S” protein or fragment thereof comprises the mutations F817P, A892P, A899P, A942P, K986P, and V987P, relative to SEQ ID NO: 14. In some embodiments, the recombinant protein comprises a sequence selected from the group consisting of SEQ ID NOs: 7-13, 19-25, and 30-31.

In another aspect of the current disclosure, pharmaceutical compositions are provided. In some embodiments, the pharmaceutical compositions comprise: (i) a SARS-CoV-2 polypeptide sequence derived from the spike (“S”) protein or a variant thereof, and (ii) one or more heterologous polypeptide sequences selected from a purification tag, a detectable label, a flexible linker, a cleavage site to allow for tag removal after purification, and a secretion signal peptide. In some embodiments, the furin site “RRAR” in the polypeptide is genetically engineered so as not to be cleaved by furin, optionally wherein the furin site is engineered to “GSAS.” In some embodiments, the SARS-CoV-2 polypeptide sequence comprises a fragment of the S protein including amino acids 319-591, or a variant thereof. In some embodiments, the detectable label comprises green fluorescent protein (GFP) or enhanced green fluorescent protein (eGFP). In some embodiments, the recombinant proteins comprise one or more mutations selected from the group consisting of: F817P, A892P, A899P, A942P, K986P, and V987P, relative to SEQ ID NO: 14. In some embodiments, the flexible linker comprises GGGGSGGGGSGG (SEQ ID NO: 34). In some embodiments, the cleavage site to allow for tag removal after purification comprises a Tobacco Etch Virus nuclear-inclusion-a endopeptidase (TEV protease) recognition sequence: GENLYFQG (SEQ ID NO: 35). In some embodiments, the secretion signal peptide comprises MFLLTTKRT (SEQ ID NO: 36). In some embodiments, the recombinant proteins further comprise a foldon trimerization domain. In some embodiments, the recombinant proteins comprise a solubility enhancer peptide comprising maltose binding protein (MBP). In some embodiments, the maltose binding protein (MBP) comprises a GGSK₁₀ sequence (SEQ ID NO: 38) at its N terminus or C terminus. In some embodiments, the heterologous polypeptide sequence comprises (a) a purification tag comprising a HIS tag; (b) a detectable label comprising Green Fluorescent Protein or enhanced Green Fluorescent Protein; (c) a flexible linker comprising GGGGSGGGGSGG (SEQ ID NO: 34); (d) a cleavage site to allow for tag removal after purification comprising a Tobacco Etch Virus nuclear-inclusion-a endopeptidase (TEV protease) recognition sequence, GENLYFQG (SEQ ID NO: 35); (e) a secretion signal peptide comprising MFLLTTKRT (SEQ ID NO: 36); (f) a foldon trimerization domain; wherein the “S” protein or fragment thereof comprises the mutations F817P, A892P, A899P, A942P, K986P, and V987P, relative to SEQ ID NO: 14. In some embodiments, the recombinant protein comprises a sequence selected from the group consisting of SEQ ID NOs: 7-13, 19-25, and 30-31. In some embodiments, the pharmaceutical compositions further comprise a pharmaceutically acceptable carrier. In some embodiments, the pharmaceutical compositions further comprise an adjuvant.

In another aspect of the current disclosure, methods of inducing an immune response against SARS-CoV-2 in a subject in need thereof are provided. In some embodiments, the methods comprise administering to the subject an effective amount of a pharmaceutical composition comprising: (i) a SARS-CoV-2 polypeptide sequence derived from the spike (“S”) protein or a variant thereof, and (ii) one or more heterologous polypeptide sequences selected from a purification tag, a detectable label, a flexible linker, a cleavage site to allow for tag removal after purification, and a secretion signal peptide. In some embodiments, the furin site “RRAR” in the polypeptide is genetically engineered so as not to be cleaved by furin, optionally wherein the furin site is engineered to “GSAS.” In some embodiments, the SARS-CoV-2 polypeptide sequence comprises a fragment of the S protein including amino acids 319-591, or a variant thereof. In some embodiments, the detectable label comprises green fluorescent protein (GFP) or enhanced green fluorescent protein (eGFP). In some embodiments, the recombinant proteins comprise one or more mutations selected from the group consisting of: F817P, A892P, A899P, A942P, K986P, and V987P, relative to SEQ ID NO: 14. In some embodiments, the flexible linker comprises GGGGSGGGGSGG (SEQ ID NO: 34). In some embodiments, the cleavage site to allow for tag removal after purification comprises a Tobacco Etch Virus nuclear-inclusion-a endopeptidase (TEV protease) recognition sequence: GENLYFQG (SEQ ID NO: 35). In some embodiments, the secretion signal peptide comprises MFLLTTKRT (SEQ ID NO: 36). In some embodiments, the recombinant proteins further comprise a foldon trimerization domain. In some embodiments, the recombinant proteins comprise a solubility enhancer peptide comprising maltose binding protein (MBP). In some embodiments, the maltose binding protein (MBP) comprises a GGSK₁₀ sequence (SEQ ID NO: 38) at its N terminus or C terminus. In some embodiments, the heterologous polypeptide sequence comprises (a) a purification tag comprising a HIS tag; (b) a detectable label comprising Green Fluorescent Protein or enhanced Green Fluorescent Protein; (c) a flexible linker comprising GGGGSGGGGSGG (SEQ ID NO: 34); (d) a cleavage site to allow for tag removal after purification comprising a Tobacco Etch Virus nuclear-inclusion-a endopeptidase (TEV protease) recognition sequence, GENLYFQG (SEQ ID NO: 35); (e) a secretion signal peptide comprising MFLLTTKRT (SEQ ID NO: 36); (f) a foldon trimerization domain; wherein the “S” protein or fragment thereof comprises the mutations F817P, A892P, A899P, A942P, K986P, and V987P, relative to SEQ ID NO: 14. In some embodiments, the recombinant protein comprises a sequence selected from the group consisting of SEQ ID NOs: 7-13, 19-25, and 30-31. In some embodiments, the pharmaceutical compositions further comprise a pharmaceutically acceptable carrier. In some embodiments, the pharmaceutical compositions further comprise an adjuvant. In some embodiments, the immune response against SARS-CoV-2 in the subject comprises a cellular immune response, a humoral immune response, or both a cellular and a humoral immune response.

In another aspect of the current disclosure, methods for identifying whether a subject has been exposed to SARS-CoV-2 are provided. In some embodiments, the methods comprise: (a) obtaining a sample from the subject; (b) contacting the sample with the recombinant protein of claim 1 under conditions that allow SARS-CoV-2 antibodies, if present in the sample, to bind to the recombinant protein and form an antibody-antigen complex; and (c) detecting the complex. In some embodiments, the complex is detected by contacting the complex with a secondary antibody that binds the complex and comprises a detectable label, optionally wherein the secondary antibody is an anti-human antibody that binds human SARS-CoV-2 antibodies and comprises a fluorometric label or colorimetric label.

### BRIEF DESCRIPTION OF THE DRAWINGS

FIG. 1A-FIG. 1B. Provides the amino acid sequences of a recombinant SARS-CoV-2 protein of the present disclosure, engineered for either cytoplasmic expression (A) (SEQ ID NO: 7), or secretion in insect cells (B) (SEQ ID NO: 8). The receptor binding domain of the S protein (S-RBD(319-591)) is in plain font; TEV protease recognition sequence is in bold font; a linker sequence is underlined; the enhanced Green Fluorescent Protein is in italics; and a His-tag is in bold, underlined font. FIG. 1B shows the secretion signal peptide for insect cells in bold italics.

FIG. 2A-FIG. 2B. Provides the amino acid sequences of a recombinant SARS-CoV-2 protein of the present disclosure, engineered for secretion in insect cells (A) (SEQ ID NO: 9). The secretion signal peptide for insect cells in bold italics; the ectodomain of the S protein (S-Ecto (1-1220) is in plain font (the modified furin recognition site GSAS (SEQ ID NO: 30) is underlined); (B) (SEQ ID NO: 10) TEV protease recognition sequence is in bold font; a linker sequence is underlined; the enhanced Green Fluorescent Protein is in italics; and a His-tag is in bold, underlined font.

FIG. 3A-FIG. 3B. Provides the amino acid sequences of a recombinant SARS-CoV-2 protein of the present disclosure, engineered for either cytoplasmic expression (A) (SEQ ID NO: 11) or secretion (B) (SEQ ID NO: 12) in insect cells. The N protein is in plain font; TEV protease recognition sequence is in bold font; a linker sequence is underlined; the enhanced Green Fluorescent Protein is in italics; and a His-tag is in bold, underlined font. FIG. 3B shows the secretion signal peptide for insect cells in bold italics.

FIG. 4. Provides the amino acid sequences of a recombinant protein of the present disclosure, engineered for expression in bacteria (SEQ ID NO: 13). A His-tag is in bold, underlined font; maltose binding protein is in italics; a linker sequence is underlined; the TEV protease recognition sequence is in bold font; the amino acid sequence of S-RBD(319-591) is in plain font.

FIG. 5. Provides the consensus amino acid sequence of the SARS-CoV-2 S protein (MN938384; protein ID QHN73795.1) (SEQ ID NO: 14). The furin site, RRAS (SEQ ID NO: 31), is in bold font.

FIG. 6A-FIG. 6C. Provides the consensus amino acid sequences of the SARS-CoV-2 N-protein (SEQ ID NO: 15); MN938384, protein ID QHN73802.1 (A); M protein (SEQ ID NO: 16); MN938384, protein ID QHN 73798.1 (B); and E protein (SEQ ID NO: 17), MN938384, protein ID AHN 73797.1 (C).

FIG. 7. Provides the amino acid sequences of a recombinant SARS-CoV-2 protein of the present disclosure, engineered for secretion in insect cells (SEQ ID NO: 18). The ectodomain of the S protein (S-Ecto) sequence is in plain font; the GSAS sequence is in bold font and shaded; the linker sequence is in bold font; the TEV sequence is underlined; MBP is in italics.

FIG. 8. Provides the amino acid sequences of a recombinant SARS-CoV-2 protein of the present disclosure, engineered for secretion in insect cells (SEQ ID NO: 19). The ectodomain of the S protein is in plain font; the GSAS (SEQ ID NO: 30) sequence is in bold font and shaded; the linker sequence is in bold font; the TEV sequence is underlined; MBP is in italics; and the GSK10 sequence is in italics and underlined.

FIG. 9. Provides the amino acid sequences of a recombinant SARS-CoV-2 protein of the present disclosure, engineered for secretion in insect cells (SEQ ID NO: 20). The receptor binding domain of the S protein (S-RBD) is in plain font; the linker sequence is in bold font; the TEV sequence is underlined; MBP is in italics.

FIG. 10. Provides the amino acid sequences of a recombinant SARS-CoV-2 protein of the present disclosure, engineered for secretion in insect cells (SEQ ID NO: 21). The receptor binding domain of the S protein (S-RBD) is in plain font; the linker sequence is in bold font; the TEV sequence is underlined; MBP is in italics; the GGSK10 sequence is in italics and underlined.

FIG. 11. Provides the amino acid sequences of a recombinant SARS-CoV-2 protein of the present disclosure, engineered for secretion in insect cells (SEQ ID NO: 22). The receptor binding domain of the S protein (S-RBD) is in plain font; the linker sequence is in bold font; the TEV sequence is underlined; eGFP is in italics.

FIG. 12. Provides the amino acid sequences of a recombinant SARS-CoV-2 protein of the present disclosure, engineered for secretion in insect cells (SEQ ID NO: 23). The ectodomain of the S protein (S-Ecto) sequence is in plain font; the GSAS sequence is in bold font and shaded; the linker sequence is in bold font; the TEV sequence is underlined; the eFGP sequence is in italics.

FIG. 13A-FIG. 13B. Provides the amino acid sequences of a recombinant SARS-CoV-2 protein of the present disclosure, engineered for secretion in insect cells. (A) (SEQ ID NO: 24) Without NT secretion signal; (B) (SEQ ID NO: 25) with NT secretion signal. For both A and B, the receptor binding domain of the S protein (S-RBD) is in plain font; the linker sequence is in bold font; the TEV sequence is underlined; eGFP is in italics. For (B), the secretion signal ins bold, italics.

FIG. 14. Provides the amino acid sequences of a recombinant SARS-CoV-2 protein of the present disclosure, engineered for bacterial cell expression (SEQ ID NO: 26). The N protein is in plain font; TEV protease recognition sequence is in bold font; a linker sequence is underlined.

FIG. 15. Provides the amino acid sequences of a recombinant SARS-CoV-2 protein of the present disclosure, engineered for bacterial cell expression (SEQ ID NO: 27). The N protein is in plain font; TEV protease recognition sequence is in bold font; a linker sequence is underlined; the MBP sequence is in italics; the GGSK10 sequence is in italics and underlined.

FIG. 16. Provides the amino acid sequences of a recombinant SARS-CoV-2 protein of the present disclosure, engineered for bacterial co-expression with CyDisCO system (SEQ ID NO: 28). The receptor binding domain of the S protein (S-RBD) is in plain font; the MBP sequence is in italics; the linker sequence is in bold font; the TEV sequence is underlined.

FIG. 17. Provides the amino acid sequences of a recombinant SARS-CoV-2 protein of the present disclosure, engineered for bacterial co-expression with CyDisCO system (SEQ ID NO: 29). The receptor binding domain of the S protein (S-RBD) is in plain font; the linker sequence is in bold font; the TEV sequence is underlined; the MBP sequence is in italics; the GGSK10 sequence (SEQ ID NO: 38) is in italics and underlined.

FIG. 18A-FIG. 18E. Domain map of full-length Spike protein and purified Spike constructs. (A) Full length SARS-CoV-2 Spike protein. SS: signal sequence; NTD: N-terminal domain; RBD: receptor binding domain; RBM: receptor binding motif; SD1/2: subdomain 1 and 2; FP: fusion peptide; HR1: heptad repeat 1; CH: central helix; CD: connector domain; HR2: heptad repeat 2; TM: transmembrane domain; CP: cytoplasmic peptide. (B) S-RBD-eGFP, (C) S-Ecto-eGFP, (D) S-Ecto-HexaPro(+F), and (E) S-Ecto-HexaPro(−F). Respective domains are insect cell Secretion Peptide (yellow), Spike protein (Blue), TEV cleavage site (grey), linker regions (magenta), foldon domain (orange), 12× His Tag (peach).

FIG. 19A-FIG. 19D. SDS-PAGE of fully purified SARS-CoV-2 Spike Ectodomains and RBD with Affinity tag and eGFP removed. Five μg of (A) S-Ecto-HexaPro(+F), (B) S-Ecto-HexaPro(−F), (C) S-RBD, and (D) S-Ecto were resolved on 4-12% SDS-PAGE.

FIG. 20A-FIG. 20B. Glycosylation of SARS-CoV-2 S-Ectodomain. (A) Schematic representation of SARS-CoV-2 S protein's N- and O-linked glycosylation sites. (B) Immunoblotting analysis of Sialidase A, O-glycanase, and N-glycanase treated S-Ectodomain.

FIG. 21A-FIG. 21B. Activity assessment of purified S-Ecto and RBD constructs for binding to hACE2 protein in SPR. (A) SPR curve-fit plots of spike ectodomain with six proline mutations, with and without a foldon domain ((S-Ecto-HexaPro(+F) and (S-Ecto-HexaPro(−F) respectively), spike ectodomain with eGFP (S-Ecto-eGFP), and spike receptor binding domain with eGFP (S-RBD-eGFP) at concentrations of 2, 10, and 50 μg/ml binding to hACE2. (B) K_{D} Values with standard deviation (SD) of each recombinant protein binding to hACE2.

FIG. 22. Functional S-Ecto-eGFP binding is reduced by ACE2 receptor blockade. Calu3 cells were incubated with 40 μg/mL α-ACE2 or goat IgG antibody (α-IgG) for 45 min prior to incubation with the S1-ectodomain-eGFP protein. Data is represented as the fold increase of GFP MFI (median fluorescent intensity) compared to GFP-His tag control (mean±SEM). N=4 independent experiments. * P<0.05.

FIG. 23. Reduced functional S-Ecto-eGFP binding to surface ACE2 in BET-inhibitor treated Calu3 cells. Calu3 cells were treated with BET inhibitors (JQ1, RVX-208; RVX) or control DMSO vehicle (VEH) for 24 h prior to incubation with the S1-ectodomain-eGFP protein. Data is represented as the fold increase of GFP MFI (median fluorescent intensity) compared to GFP-His tag control (mean±SEM). N=3-4 independent experiments. * P<0.05,** P<0.001.

FIG. 24. Provides the amino acid sequences of a recombinant protein of the present disclosure (SEQ ID NO: 30). A His-tag is in bold, underlined font; maltose binding protein is in italics; a linker sequence is underlined; the TEV protease recognition sequence is in bold font; the amino acid sequence of S-RBD(319-591) is in plain font.

FIG. 25. Provides the amino acid sequences of a recombinant protein of the present disclosure (SEQ ID NO: 31). A His-tag is in bold, underlined font; maltose binding protein is in italics; a linker sequence is underlined; the TEV protease recognition sequence is in bold font; the amino acid sequence of S-RBD(319-591) is in plain font.

### DETAILED DESCRIPTION

Disclosed are compositions, vectors, kits, data analyses, and methods for inducing an immune response against Severe Acute Respiratory Syndrome Coronavirus 2 (SARS-CoV-2), and/or for detecting exposure to SARS-CoV-2, which may be described herein using definitions as set forth below and throughout the application.

Unless otherwise specified or indicated by context, the terms “a,” “an,” and “the,” mean “one or more.” For example, “protein” or “domain” should be interpreted to mean “one or more proteins” and “one or more domains,” respectively.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “subject,” “host,” or “individual” or “patient” typically refer to an animal at risk for acquiring an infection by SARS-CoV-2, such as a human. The terms “patient,” “subject,” “host,” or “individual” may be used interchangeably.

As used herein, an “immune response” may include an antibody response (i.e., a humoral response), where an immunized individual is induced to produce antibodies against an administered antigen (e.g., IgY, IgA, IgM, IgG, or other antibody isotypes). As used herein, an “immune response” also may include a cell-mediated response, for example, a cytotoxic T-cell response against cells expressing foreign peptides derived from an administered antigen in the context of a major histocompatibility complex (MHC) class I molecule.

As used herein, “potentiating” or “enhancing” an immune response means increasing the magnitude and/or the breadth of the immune response. For example, the number of cells that recognize a particular epitope may be increased (“magnitude”) and/or the numbers of epitopes that are recognized may be increased (“breadth”).

As used herein the term “sample,” with reference to a patient sample, or a subject sample, refers to a biological sample from a subject or patent, and such samples include, but are not necessarily limited to bodily fluids such as saliva, urine and blood-related samples (e.g., whole blood, serum, plasma, and other blood-derived samples), cerebral spinal fluid, bronchoalveolar lavage, stool, nasal swab, and the like. In some embodiments, the biological sample is a skin sample. Biological samples can be obtained by any known means including needle stick, needle biopsy, swab, and the like. A biological sample may be fresh or stored (e.g. blood or blood fraction stored in a blood bank). Samples can be stored for varying amounts of time, such as being stored for an hour, a day, a week, a month, or more than a month. The biological sample may be a bodily fluid expressly obtained for the assays disclosed herein, or a bodily fluid obtained for another purpose which can be sub-sampled in order to carry out the method. In some embodiments, the sample contains antibodies, such as antibodies against a virus with which the patient or the subject is infected.

As used herein, “viral load” is the amount of virus present in a sample from a subject infected with the virus. Viral load is also referred to as viral titer or viremia. Viral load can be measured in variety of standard ways including copy Equivalents of the viral RNA (vRNA) genome per milliliter individual sample (vRNA copy Eq/ml). This quantity may be determined by standard methods that include RT-PCR.

Severe Acute Respiratory Syndrome Coronavirus 2

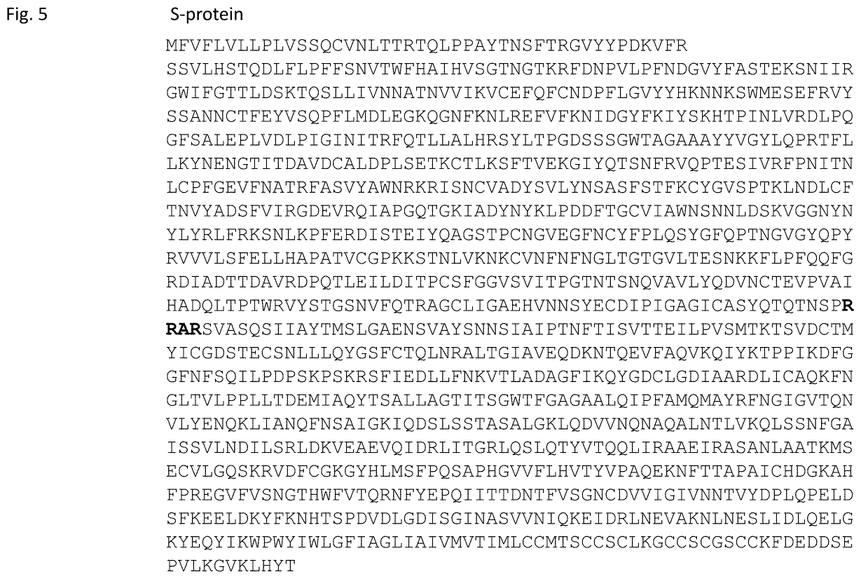

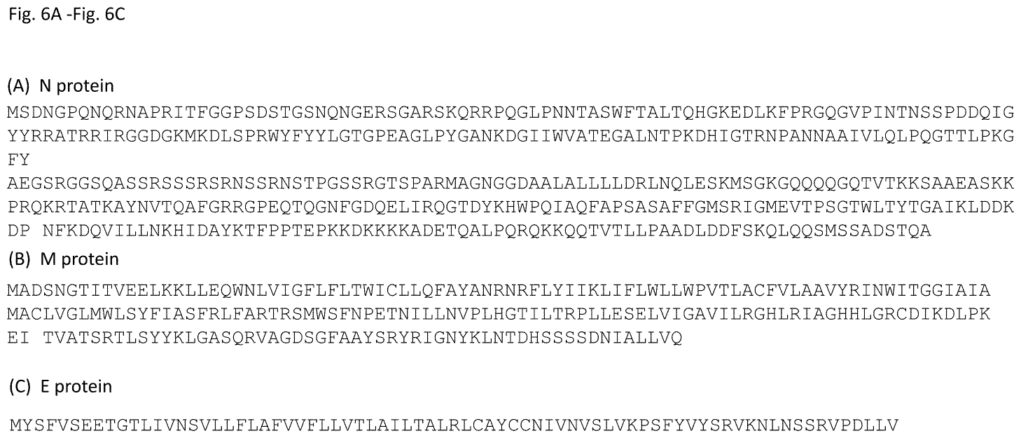

As used herein, the term Severe Acute Respiratory Syndrome Coronavirus 2 (SARS-CoV-2) refers to an enveloped, non-segmented, positive sense RNA virus that is included in the sarbecovirus, ortho corona virinae subfamily which is broadly distributed in humans and other mammals. Its diameter is about 65-125 nm, containing single strands of RNA and provided with crown-like spikes on the outer surface. A nucleic acid sample isolation from pneumonia patients who were some of the workers in the Wuhan seafood market found that strains of SARS-CoV-2 had a length of 29.9 kb. Structurally, SARS-CoV-2 has four main proteins including spike (S) glycoprotein, small envelope (E) glycoprotein, membrane (M) glycoprotein, and nucleocapsid (N) protein, and also several accessory proteins. The S, E, and M proteins together create the viral envelope, while the N protein holds the RNA genome. (See e.g., FIGS. 5 and 6 for the amino acid sequences of S (SEQ ID NO: 14), E (SEQ ID NO: 17), M (SEQ ID NO: 16), and N (SEQ ID NO: 15)) proteins.

The name “coronavirus” is derived from the Latin word “corona” meaning crown or halo, and refers to the characteristic appearance of the virus under an electron microscopy, where the virus includes a fringe of large, bulbous surface projections creating an image reminiscent of a crown or halo. This coronal morphology is created by the viral spike protein (S), which is present on the surface of the virus. The spike or S glycoprotein is a transmembrane protein with a molecular weight of about 150 kDa found on the outer portion of the virus and is 1273 amino acids in length (see e.g., FIG. 5). S protein forms homotrimers protruding from the viral surface and facilitates binding of viruses to host cells by recognition of the angiotensin-converting enzyme 2 receptor (ACE2). This protein is widely found in different organs such as the lung, kidney, heart, and endothelial tissue. Therefore, patients who are infected with this virus not only experience respiratory problems such as pneumonia leading to Acute Respiratory Distress Syndrome (ARDS), but also experience disorders of the heart, kidneys, and digestive tract. The S glycoprotein includes a furin cleavage site (see “RRAS” (SEQ ID NO: 33) in bold font in FIG. 5), and the S protein is cleaved within the host cell by furin-like proteases into 2 subunits, 51 and S2. 51 is responsible for the determination of the host virus range and cellular tropism with the receptor binding domain make-up, while S2 functions to mediate virus fusion to host cells. The S2 domain transverses the viral membrane and includes an N-terminal ectodomain and a cytosolic C-terminus. The 51 domain associates non-covalently with the ectodomain of S2.

The nucleocapsid protein, known as N protein, is 419 amino acids in length (see e.g., FIG. 6A (SEQ ID NO: 15)) and is bound to the nucleic acid material of the virus. Because the protein is bound to RNA, the protein is involved in processes related to the viral genome, the viral replication cycle, and the cellular response of host cells to viral infections.

The membrane protein, or M protein, is 218 amino acids in length (see e.g., FIG. 6B (SEQ ID NO: 16)) and plays a role in determining the shape of the virus envelope. In addition, binding with M protein helps to stabilize N proteins and promotes completion of viral assembly by stabilizing N protein-RNA complex inside the virion.

The envelope or E protein is the smallest structural protein in SARS-CoV-2, including only 75 amino acids (see e.g., FIG. 6C (SEQ ID NO: 17)), and plays a role in the production and maturation of the virus.

Nucleic Acids, Polypeptides, Proteins, and Peptides

The terms “polynucleotide,” “nucleic acid” and “nucleic acid sequence” refer to a polymer of DNA or RNA nucleotide of genomic or synthetic origin (which may be single-stranded or double-stranded and may represent the sense or the antisense strand). The polynucleotides contemplated herein may encode and may be utilized to express one or more SARS-CoV-2 polypeptides such as the disclosed recombinant proteins of SARS-CoV-2.

As used herein, polypeptide, proteins, and peptides comprise polymers of amino acids, otherwise referred to as “amino acid sequences.” A polypeptide or protein is typically of length >100 amino acids (Garrett & Grisham, Biochemistry, 2^{nd} edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). However, the terms “polypeptide,” “protein,” and “peptide” may be used interchangeably herein.

As contemplated herein, a polypeptide, protein, or peptide may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine). In some embodiments, the disclosed recombinant proteins of SARS-CoV-2 may be modified to include a non-naturally occurring N-terminal modification such as an acetylation. In some embodiments, the disclosed recombinant proteins of SARS-CoV-2 may be modified to include a non-naturally occurring C-terminal modification such as an amidation.

The amino acid sequences contemplated herein may include one or more amino acid substitutions relative to a reference amino acid sequence (e.g., relative to any the sequences in FIGS. 5 and 6). In some cases, these substitutions may be conservative amino acid substitutions relative to the reference amino acid sequence. For example, a variant, mutant, or derivative polypeptide may include conservative amino acid substitutions relative to a reference polypeptide (e.g., relative to any of sequences shown in FIGS. 5 and 6). “Conservative amino acid substitutions” are those substitutions that are predicted to interfere least with the properties of the reference polypeptide. In other words, conservative amino acid substitutions substantially conserve the structure and the function of the reference protein. Table 1 provides a list of exemplary conservative amino acid substitutions.

TABLE 1

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. In contrast, non-conservative amino acid substitutions generally disrupt and/or alter (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in a reference amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence that extends the half-life of the fusion polypeptide in serum. A “variant” of a reference polypeptide sequence may include a fusion polypeptide comprising the reference polypeptide. In some embodiments, the disclosed recombinant SARS-CoV-2 proteins may be defined as fusion polypeptides that include SARS-CoV-2 amino acid sequences optionally fused to non-SARS-CoV-2 amino acid sequences (i.e., heterologous amino acid sequences).

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, 300, 350, 400, 450, 500, 550, 600, 650, 700, 750, 800, 850, 900, 950, or 1000 contiguous amino acid residues of a reference polypeptide; or a fragment may comprise no more than 5, 10, 15, 20, 25, 30, 40, 50, 60, 70, 80, 90, 100, 150, 250, 300, 350, 400, 450, 500, 550, 600, 650, 700, 750, 800, 850, 900, 950, or 1000 contiguous amino acid residues of a reference polypeptide; or a fragment may comprise a range of contiguous amino acid residues of a reference polypeptide bounded by any of these values (e.g., 50-100 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of amino acid residue matches between at least two polypeptide sequences aligned using a standardized algorithm. Methods of polypeptide sequence alignment are well-known. Some alignment methods take into account conservative amino acid substitutions, non-conservative amino acid substitutions, deletions, and/or insertions. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence as defined by a particular SEQ ID number, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 contiguous amino acid residues of any of, for example, the sequences shown in FIGS. 5 and 6; or a fragment of no more than 15, 20, 30, 40, 50, 100, 150, 200, 250, 300, 350, 400, 450, 500, 550, 600, 650, at least 700, at least 750, at least 800, at least 850, at least 900, at least 950, or at least 1000 contiguous amino acid residues of any of the sequences shown in FIG. 5 or 6; or over a range bounded by any of these values (e.g., a range of 50-100, 100-200, etc. amino acid residues). Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In other embodiments, a pair of polypeptides may show, for example, at least 20%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

In some embodiments, a peptide of the present disclosure comprises a variant of a fragment of a SARS-CoV-2 protein. By way of example, but not by way of limitation, a fragment may comprise an S protein RBD, an S protein ectodomain, or another S protein fragment. In some embodiments, a fragment may comprise variants. By way of example But not by way of limitation, variant peptides may comprise a variant of the SARS-CoV-2 S protein with the mutations F817P, A892P, A899P, A942P, K986P, and V987P, relative to SEQ ID NO: 14, also referred to herein as the “hexapro” variant because of the six proline substitution mutations.

The disclosed variants and mutants of a reference polypeptide may possess one or more biological activities associated with the reference polypeptide, or alternatively, the disclosed variants and mutants of a reference polypeptide may lack one or more biological activities associated with the reference polypeptide. For example, the disclosed recombinant SARS-CoV-2 proteins may possess one or more biological activities associated with the wild-type protein, or the disclosed recombinant proteins may lack one or more biological activities associated with the wild-type protein.

SARS-CoV-2 Recombinant Proteins

Disclosed herein are recombinant proteins comprising one or more of the SARS-CoV-2 S, N, M, and/or E proteins, variants, or fragments thereof. In some embodiments, the viral proteins, variants, or fragments thereof are fused to one or more heterologous polypeptides, and are expressed from codon-optimized nucleic acid sequences, yielding antigenic proteins that are easily produced in large quantities, and that are easily isolated and purified. Thus, in some embodiments, a recombinant protein of the present disclosure comprises (a) one or more viral sequence; and (b) one or more heterologous sequence.

A. SARS-CoV-2 Proteins, Variants, or Fragments Thereof (“Viral Sequence”)

In some embodiments, a recombinant protein of the present disclosure comprises a viral sequence comprising or consisting of the spike protein (S protein) of SARS-CoV-2. In some embodiments, the viral sequence of the recombinant protein comprises the receptor binding domain (RBD) of the S protein, and comprises or consists of amino acids 319-591 of the S protein (see e.g., the S protein as shown in FIG. 5 (SEQ ID NO: 14)). In some embodiments, the viral sequence of the recombinant protein comprises the ectodomain of the S protein and comprises or consists of amino acids 1-1220 of the S protein (e.g., the S protein as shown in FIG. 5). In some embodiments, the viral sequence of the recombinant protein comprises or consists of the N protein of SARS-CoV-2 (e.g., the N protein as shown in FIG. 6A (SEQ ID NO: 15)).

In some embodiments, the furin recognition site of the S protein is modified, e.g., is changed from RRAR (SEQ ID NO: 33) to e.g., GSAS (SEQ ID NO: 32), to avoid cleavage of the S protein by furin protease in the endoplasmic reticulum during protein production.

In some embodiments, the S protein, or a fragment thereof, comprises the mutations F817P, A892P, A899P, A942P, K986P, and V987P, relative to SEQ ID NO: 14, also referred to herein as the “hexapro” variant because of the six proline substitution mutations.

B. Heterologous Polypeptides

In some embodiments, the recombinant polypeptides disclosed herein include, in addition to one or more viral sequences (e.g., a SARS-CoV-2 protein, variant, or fragment thereof), one or more heterologous polypeptides. By way of example, but not by way of limitation, in some embodiments a recombinant polypeptide includes one or more of a purification tag (e.g., a HIS tag), a detectable label (e.g., Green Fluorescent Protein or enhanced Green Fluorescent Protein), a flexible linker (e.g., GGGGSGGGGSGG (SEQ ID NO: 34)), and a cleavage site to allow for tag removal after purification (e.g., a Tobacco Etch Virus nuclear-inclusion-a endopeptidase (TEV protease) recognition sequence, such as GENLYFQG (SEQ ID NO: 35)), a secretion signal peptide (e.g., MFLLTTKRT (SEQ ID NO: 36) secretion signal peptide for insect cells), and a solubility enhancer peptide (e.g., maltose binding protein (MBP). In some embodiments, the maltose binding protein (MBP) comprises a GGSK₁₀ sequence (SEQ ID NO: 38) at its N terminus or C terminus.

The recombinant proteins disclosed herein may include a detectable marker. Exemplary detectable markers include, but are not limited to Green Fluorescent Protein, enhanced Green Fluorescent Protein.

The disclosed recombinant proteins may include an amino acid tag sequence, for example, which may be utilized for purifying and or identifying the recombinant proteins. Suitable amino acid tag sequences may include, but are not limited to, a histidine tag sequences comprising 5-15 histidine residues, Strep-tag, chitin binding protein (CBP), maltose binding protein (MBP), and glutathione-S-transferase (GST).

The recombinant proteins disclosed herein may include a spacer or linker sequence. Suitable spacer or linker sequences may include, but are not limited to, amino acid sequences of 5, 6, 7, 8, 9, 10, 11, 12, 13, 14, 15, 20, 25, 30, 35, 40, 45, or 50 amino acids or more, or a range bounded by any of these values (e.g., a spacer of 5-15 amino acids). In some embodiments, the spacer sequence comprises only glycine and/or serine residues or is rich in glycine residues and/or serine residues and/or alanine residues. For example, in some embodiments, the spacer sequence comprises at least about 50% glycine and/or serine residues, or at least about 60%, 70%, 80%, 90%, or 95% glycine and/or serine residues. Exemplary spacer or linker sequences include, but are not limited to: GGGGSGGGGSGG (SEQ ID NO: 34).

The recombinant proteins disclosed herein may include one or more protease recognition sequences. Such sequences may be used to remove portions of the recombinant protein, for example, after protein isolation, and to aid in protein purification. For example, a protease recognition sequence, positioned between the SARS-CoV-2 protein and a detectable marker or a purification tag, would allow for removal of the marker or tag, if so desired, by simply subjecting the recombinant protein to protease digestion with the correct protease. Exemplary protease recognition sequences include, but are not limited to: TEV and thrombin.

The recombinant protein disclosed herein may include a secretion signal, for example, to facilitate isolation of the recombinant protein from a host cell. Secretion signals are well known in the art and signals for bacterial, insect, and/or mammalian cells may be incorporated into the recombinant proteins. Exemplary secretion signals include, but are not limited to: MFLLTTKRT (SEQ ID NO: 36), a secretion signal for insect cells.

The recombinant protein disclosed herein may include a solubility enhancer to facilitate isolation and purification of the protein. Exemplary solubility enhancer peptides include but are not limited to: MBP.

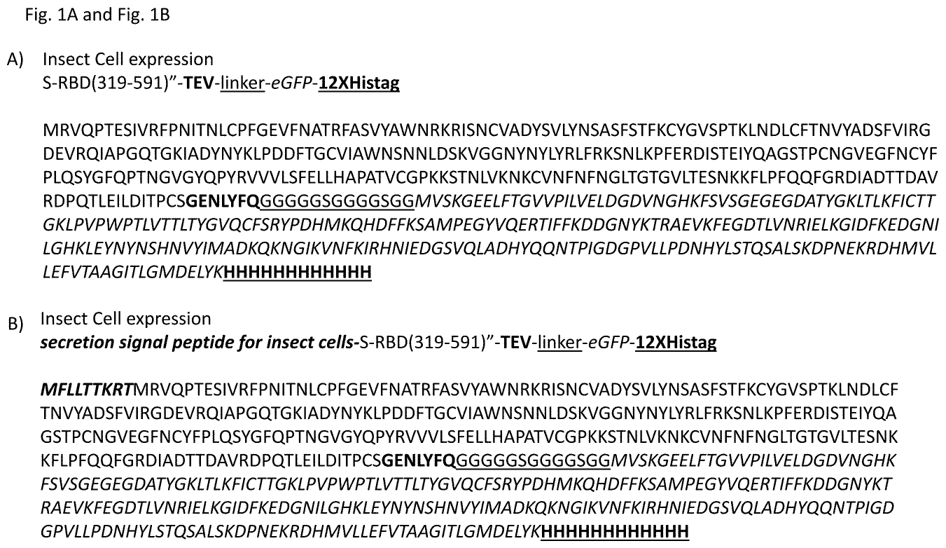

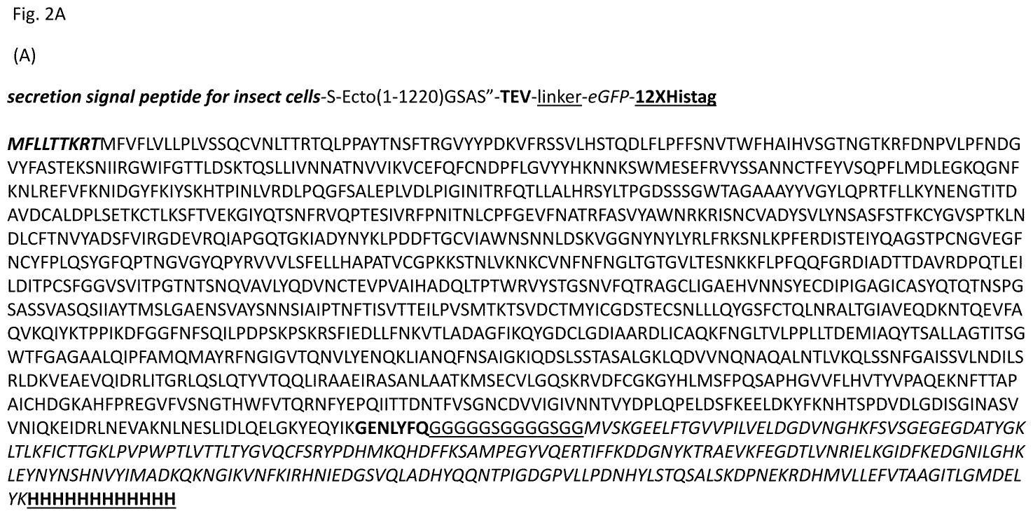

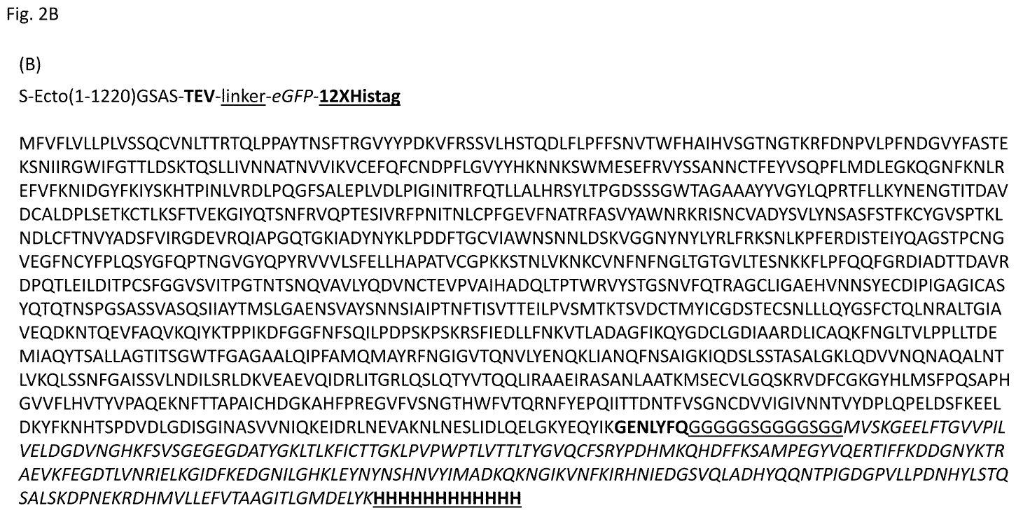

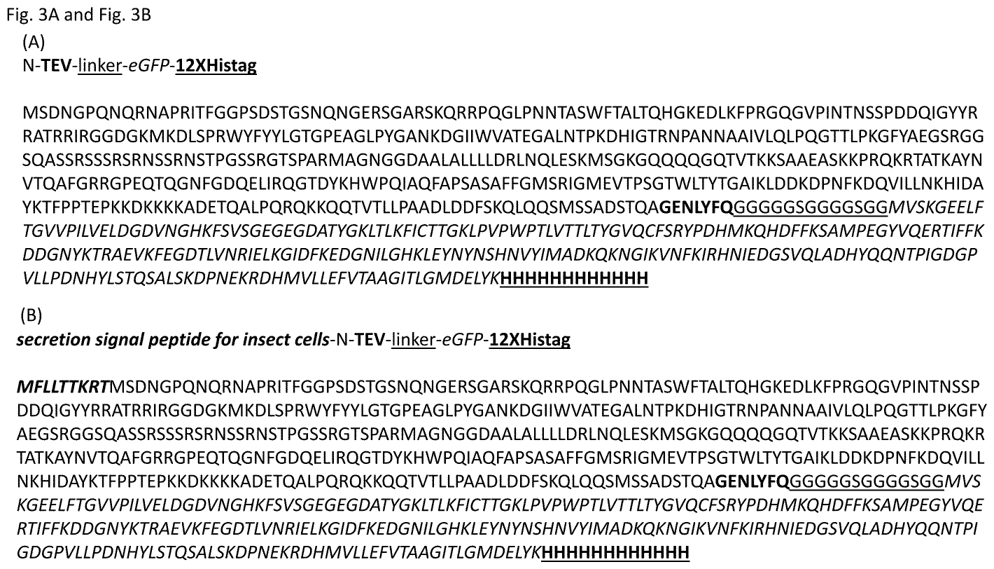

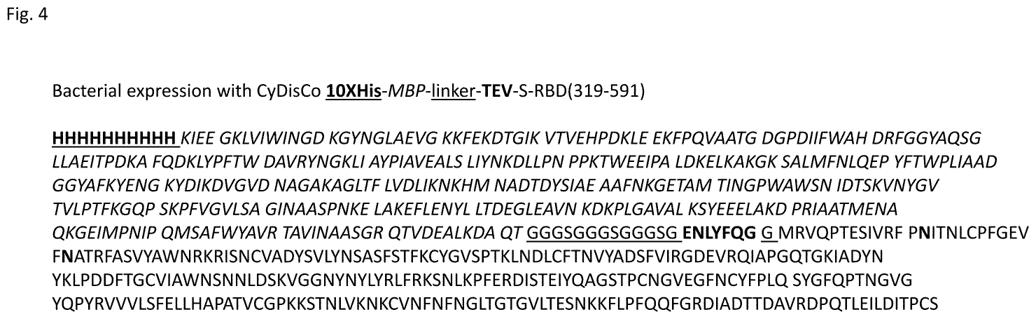

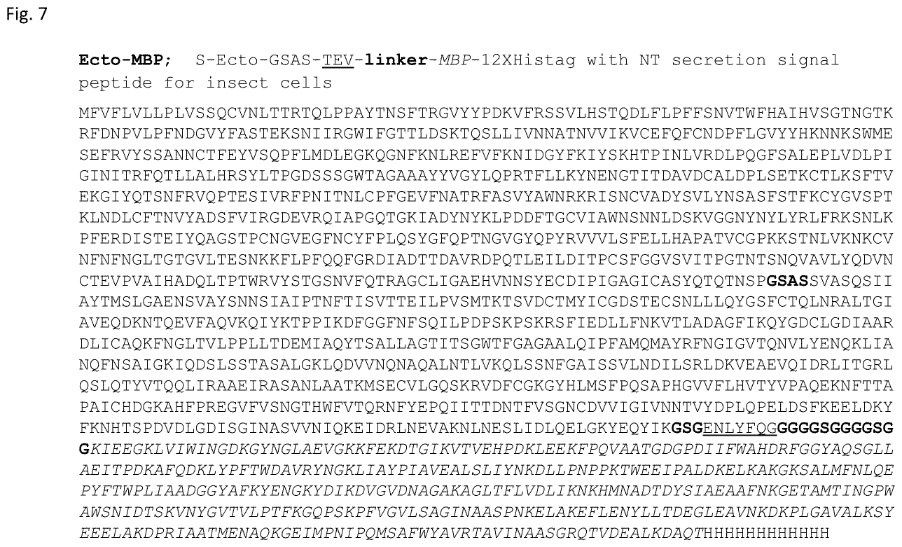

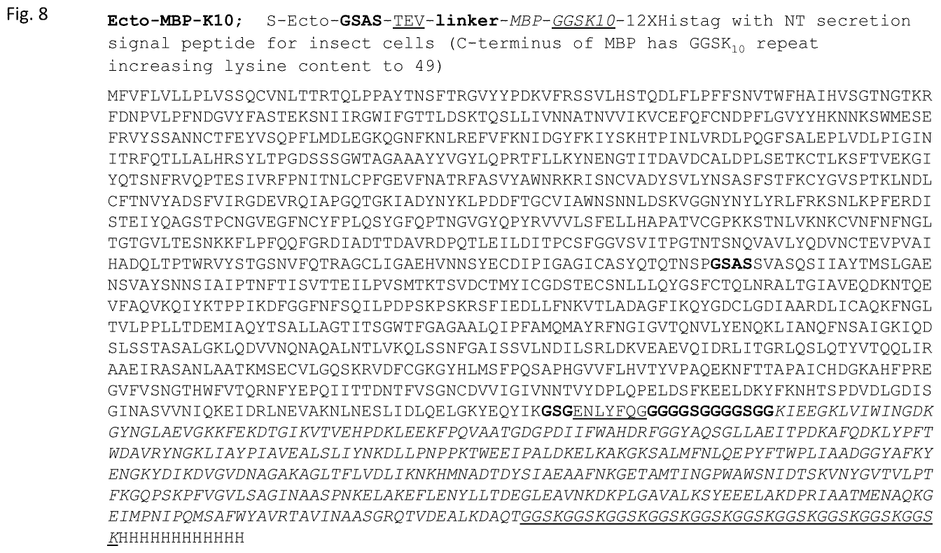

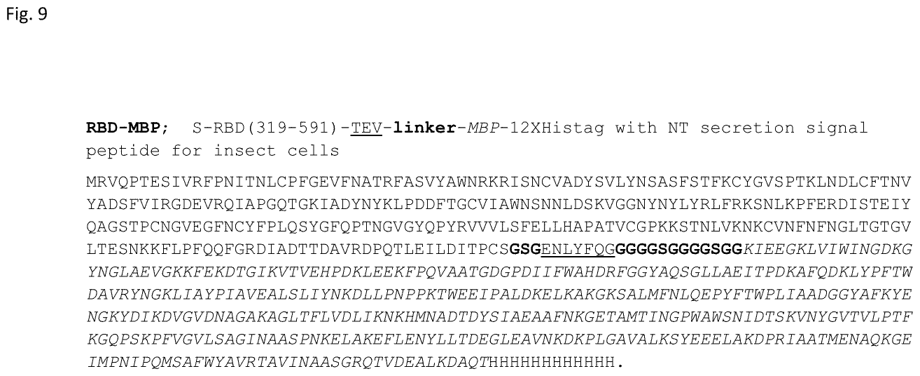

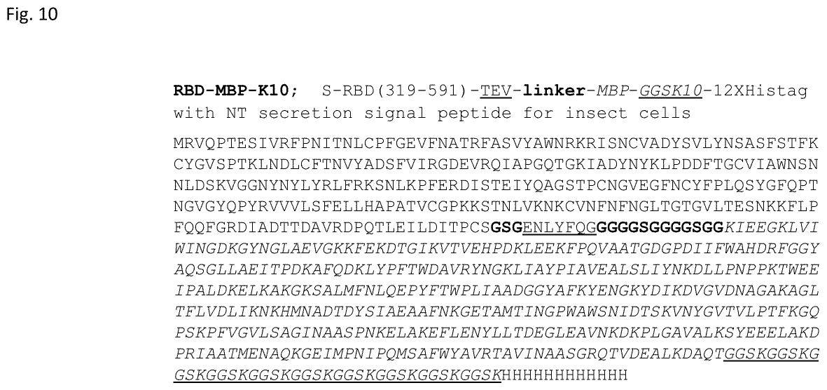

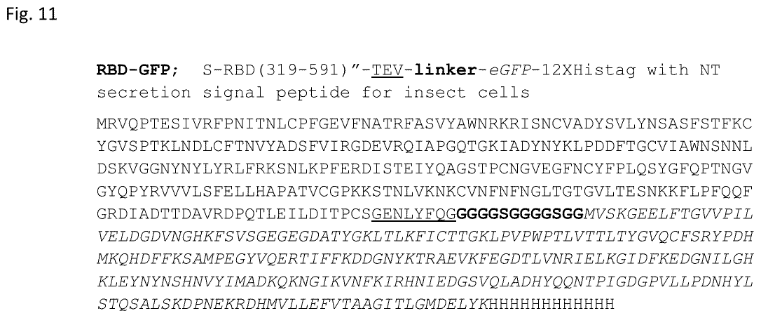

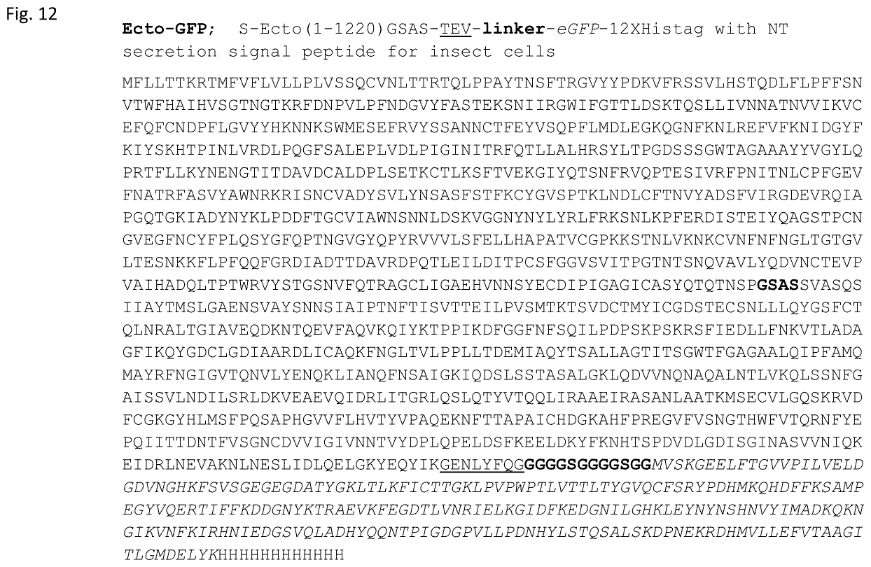

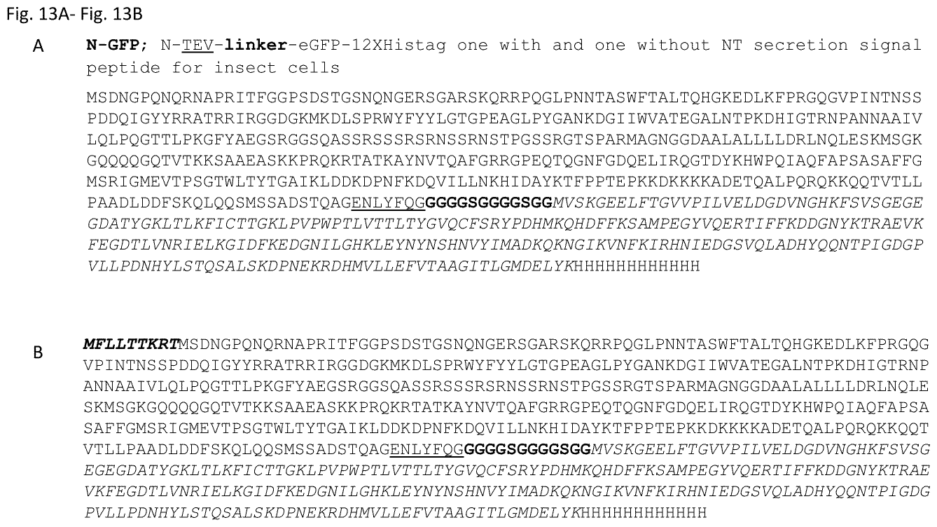

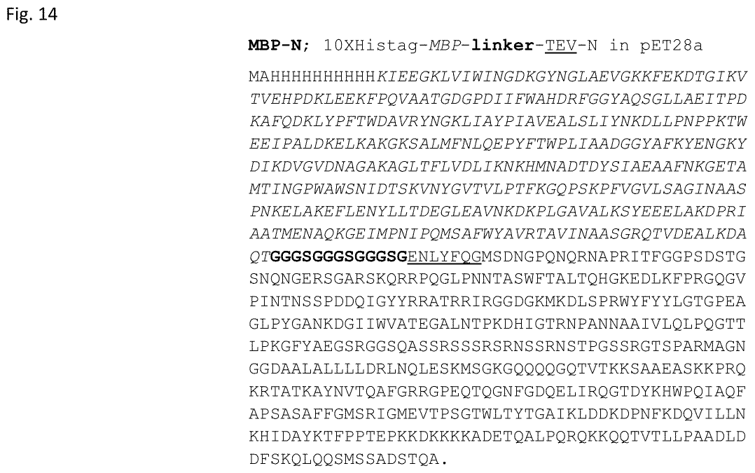

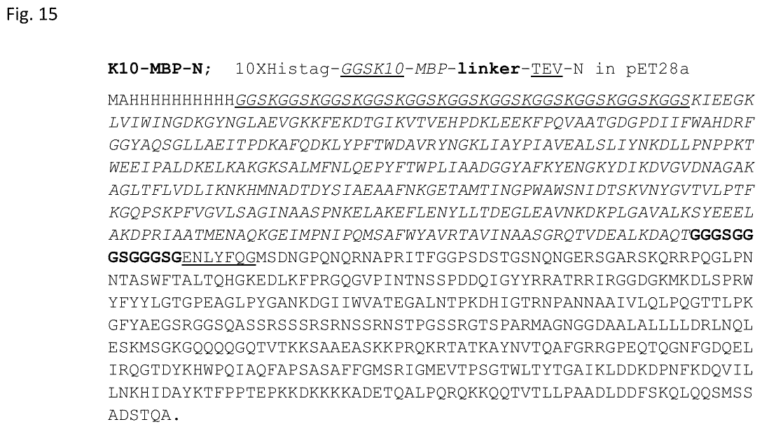

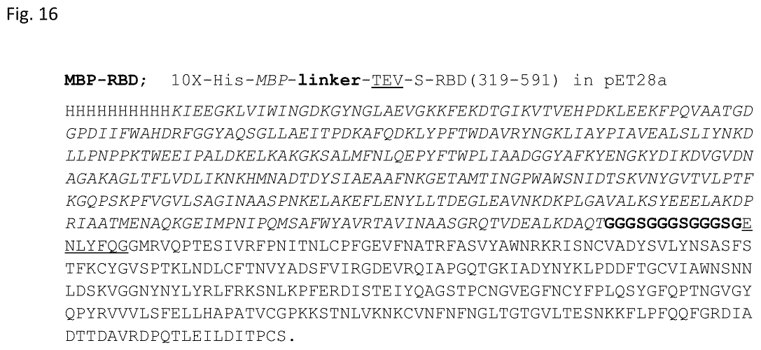

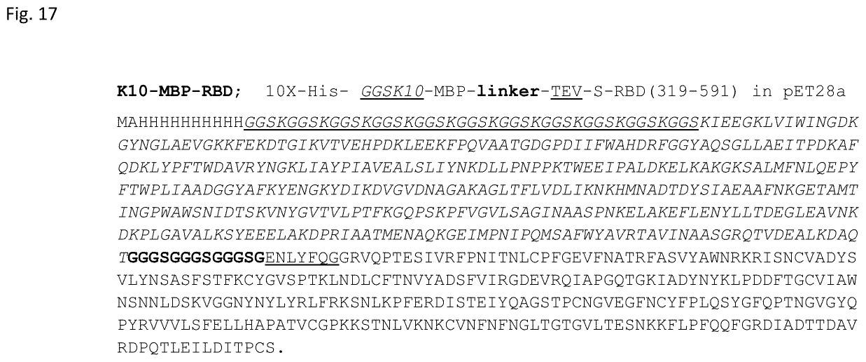

Exemplary recombinant protein sequences are provided in FIGS. 1-4. Corresponding DNA sequences are shown in the Sequence Listing provided herewith.

| Type | Name | Comments |
|---|---|---|
| Amino acid | Surface glycoprotein | Reference sequence |
| sequence | S-protein | Accession no: MN938384 |
| | SARS-CoV-2 | Protein ID: QHN73795.1 |
| | | FIG. 5 (SEQ ID NO: 14) |
| Amino acid | Envelope protein | Reference sequence |
| sequence | E-protein | Accession no: MN938384 |
| | SARS-CoV-2 | Protein ID: QHN73797.1 |
| | | FIG. 6C (SEQ ID NO: 17) |
| Amino acid | Membrane glycoprotein | Reference sequence |
| sequence | M-protein | Accession no: MN938384 |
| | SARS-CoV-2 | Protein ID: QHN73798.1 |
| | | FIG. 6B (SEQ ID NO: 16) |
| Amino acid | Nucleocapsid phosphoprotein | Reference sequence |
| sequence | N-protein | Accession no: MN938384 |
| | SARS-CoV-2 | Protein ID: QHN73802.1 |
| | | FIG. 6A (SEQ ID NO: 15) |
| Amino acid | Recombinant: receptor binding | S-RBD (319-591)-TEV-Linker- |
| sequence | domain of S-protein | eGFP-12XHistag |
| | | FIG. 1 (SEQ ID NO: 7) |
| Amino acid | Recombinant: ectodomain of S- | S-Ecto (1-1220)GSAS-TEV-linker- |
| sequence | protein | eGFP-12XHistag with N-terminal |
| | | (NT) secretion signal peptide for |
| | | insect cells |
| | | FIG. 2 (SEQ ID NO: 9) |
| Amino acid | Recombinant: N-protein | N-TEV-linker-eGFP-12XHistag |
| sequence | | FIG. 3 (SEQ ID NO: 11) |
| Amino acid | Recombinant: receptor binding | 10XHistag-MBP-linker-TEV-S-RBD |
| sequence | domain of S-protein | (319-591) |
| | | FIG. 4 (SEQ ID NO: 13) |
| DNA sequence | Recombinant: receptor binding | S-RBD(319-591)_TEV-linker-eGFP- |
| | domain of S-protein | 12XHistag.txt |
| | | Listing (SEQ ID NO: 2) |
| DNA sequence | Recombinant: ectodomain of S- | S-Ecto(1-1220)GSAS-TEV-linker- |
| | protein | eGFP-12Xhistag_Fasta.txt |
| | | Listing (SEQ ID NO: 1) |
| DNA sequence | Recombinant: N-protein | N-TEV-linker-eGFP- |
| | | 12XHistag_Fasta.txt |
| | | Listing (SEQ ID NO: 5) |
| DNA sequence | Recombinant: receptor binding | 10X-His-MBP-linker-TEV-S- |
| | domain of S-protein | RBD(319-591)_FASTA.txt |
| | | Listing (SEQ ID NO: 4) |
| Amino Acid | Recombinant: ectodomain of S- | Ecto-MBP; S-Ecto-GSAS-TEV- |
| Sequence | protein | linker-MBP-12XHistag with NT |
| | | secretion signal peptide for insect |
| | | cells |
| | | FIG. 7 (SEQ ID NO: 18) |
| Amino Acid | Recombinant: ectodomain of S- | Ecto-MBP-K10; S-Ecto-GSAS-TEV- |
| Sequence | protein | linker-MBP-GGSK10-12XHistag |
| | | with NT secretion signal peptide for |
| | | insect cells (C-terminus of MBP has |
| | | GGSK₁₀ repeat increasing lysine |
| | | content to 49) |
| | | FIG. 8 (SEQ ID NO: 19) |
| Amino Acid | Recombinant: receptor binding | RBD-MBP; S-RBD(319-591)-TEV- |
| Sequence | domain of S-protein | linker-MBP-12XHistag with NT |
| | | secretion signal peptide for insect |
| | | cells |
| | | FIG. 9 (SEQ ID NO: 20) |
| Amino Acid | Recombinant: receptor binding | RBD-MBP-K10; S-RBD(319-591)- |
| Sequence | domain of S-protein | TEV-linker-MBP-GGSK10- |
| | | 12XHistag with NT secretion signal |
| | | peptide for insect cells |
| | | FIG. 10 (SEQ ID NO: 21) |
| Amino Acid | Recombinant: receptor binding | RBD-GFP; S-RBD(319-591)”-TEV- |
| Sequence | domain of S-protein | linker-eGFP-12XHistag with NT |
| | | secretion signal peptide for insect |
| | | cells |
| | | FIG. 11 (SEQ ID NO: 22) |
| Amino Acid | Recombinant: ectodomain of S- | Ecto-GFP; S-Ecto(1-1220)GSAS- |
| Sequence | protein | TEV-linker-eGFP-12XHistag with |
| | | NT secretion signal peptide for insect |
| | | cells |
| | | FIG. 12 (SEQ ID NO: 23) |
| Amino Acid | Recombinant: N-protein | N-GFP; N-TEV-linker-eGFP- |
| Sequence | | 12XHistag one with and one without |
| | | NT secretion signal peptide for insect |
| | | cells |
| | | FIG. 13 (SEQ ID NOs: 24 and 25) |
| Amino Acid | Recombinant: N-protein | MBP-N; 10XHistag-MBP-linker- |
| Sequence | | TEV-N in pET28a |
| | | FIG. 14 (SEQ ID NO: 26) |
| Amino Acid | Recombinant: N-protein | K10-MBP-N; 10XHistag-GGSK10- |
| Sequence | | MBP-linker-TEV-N in pET28a |
| | | FIG. 15 (SEQ ID NO: 27) |
| Amino Acid | Recombinant: receptor binding | MBP-RBD; 10X-His-MBP-linker- |
| Sequence | domain of S-protein | TEV-S-RBD(319-591) in pET28a |
| | | FIG. 16 (SEQ ID NO: 28) |
| Amino Acid | Recombinant: receptor binding | K10-MBP-RBD; 10X-His- GGSK10- |
| Sequence | domain of S-protein | MBP-linker-TEV-S-RBD(319-591) |
| | | in pET28a |
| | | FIG. 17 (SEQ ID NO: 29) |

Expression of Recombinant Proteins

A. Vectors

The term “vector” refers to some means by which DNA or RNA encoding a protein of interest can be introduced into a host cell and expressed. There are various types of vectors including viral, plasmid, bacteriophage, cosmids, and bacterial. As used herein, a “vector” refers to a nucleic acid that has been engineered to express a heterologous polypeptide (e.g., a recombinant SARS-CoV-2 protein as disclosed herein). The vector typically includes cis-acting elements for expression of the heterologous polypeptide. Exemplary, non-limiting vectors include pVL1393 and pET28. Additional vectors, with cis-acting elements to direct expression in different host cells (e.g., insect host cells, bacterial host cells, mammalian host cells), are well known in the art.

B. Codon Optimization

The recombinant proteins expressed in the vectors disclosed herein may have the native polynucleotide sequence of a SARS-CoV-2 protein or may have a polynucleotide sequence that has been modified. For example, the presently disclosed vectors may express polypeptides from polynucleotides that encode the polypeptides where the polynucleotides contain codons that are optimized for expression in a particular host. For example, presently disclosed vectors may include one or more polypeptides from SARS-CoV-2 where the encoding polynucleotide sequence is optimized to include codons that are most prevalent in bacterial cells, insect cells, or mammalian cells. Codon usage for these organisms has been reported, and is well known in the art. Accordingly, a polynucleotide encoding the amino acid sequence of any the sequences shown in FIGS. 1-4 (SEQ ID NOs: 7-13) and 7-17 (SEQ ID NOs: 18-29), or SEQ ID NOs: 30-31. is contemplated herein wherein the polynucleotide's nucleic acid sequence has been codon-optimized for expression in bacterial cells, mammalian cells, or insect cells.

C. Protein Isolation

Methods of isolating recombinant proteins, expressed by cell culture (e.g., bacterial, insect, or mammalian cell culture) are well known in the art. By way of example, recombinant proteins expressing a His-tag can be isolated using commercially available kits (see e.g., Qiagen, Sigma, Thermo Scientific, and others), and by Ni²⁺ chromatography. Recombinant proteins including a maltose binding protein (MBP) can be isolated by amylose affinity chromatography. For bacterial cell expression of proteins including disulfide bonds, the CyDisCo system, which is based on co-expression of a protein of interest along with a sulfhydryl oxidase and a disulfide bond isomerase to produce disulfide bonded proteins in the presence of intact reducing pathways in the cytoplasm, may be employed (see e.g., Matos C F, Robinson C, Alanen H I, et al. Efficient export of prefolded, disulfide-bonded recombinant proteins to the periplasm by the Tat pathway in Escherichia coli CyDisCo strains. Biotechnol Prog. 2014; 30(2):281-290. doi:10.1002/btpr.1858; Ga̧ciarz, A., Khatri, N. K., Velez-Suberbie, M. L. et al. Efficient soluble expression of disulfide bonded proteins in the cytoplasm of Escherichia coli in fed-batch fermentations on chemically defined minimal media. Microb Cell Fact 16, 108 (2017), incorporated herein by reference).

Recombinant proteins may be further processed by treatment with a selected protease. For example, a recombinant protein may include a protease recognition site between a SARS-CoV-2 protein sequence and a detectable marker, or a His tag. Contacting the recombinant protein with the proper protease (i.e., a protease that acts on the protease recognition site), under appropriate reaction conditions will result in cleavage of the recombinant protein at the protease recognition site, thereby separating the detectable marker or His tag from the SARS-CoV-2 protein sequence.

Pharmaceutical Compositions

The compositions disclosed herein may include pharmaceutical compositions comprising the presently disclosed SARS-CoV-2 recombinant proteins formulated for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants, as are known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”)).

Formulation of the Pharmaceutical Compositions

Compositions comprising the disclosed recombinant proteins are contemplated herein. For example, pharmaceutical compositions and vaccines are contemplated herein. The disclosed recombinant proteins may be formulated as vaccine composition for administration to a subject in need thereof. Such compositions can be formulated and/or administered in dosages and by techniques well known to those skilled in the medical arts taking into consideration such factors as the age, sex, weight, and condition of the particular subject, and the route of administration.

The compositions may include pharmaceutical solutions comprising carriers, diluents, excipients (e.g., powder excipients such as lactose, sucrose, and mannitol), and surfactants (e.g., non-ionic surfactants such as Kolliphor HS 15, Kollidon 12 PF, and Tween-20), as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered prophylactically or therapeutically. In prophylactic administration, a pharmaceutical composition may be administered as a vaccine in an amount sufficient to induce an immune response for protecting against infection. In therapeutic applications, a pharmaceutical composition may be administered as a vaccine to a subject in an amount sufficient to elicit a therapeutic effect (e.g., an immune response to the administered antigen, which cures or at least partially arrests or slows symptoms and/or complications of disease (i.e., a “therapeutically effective dose”)). Inducing a protective response may include inducing sterilizing immunity against a pathogen (e.g., against SARS-CoV-2). Inducing a therapeutic response may include reducing the pathogenic load of a subject, for example, as determined by measuring the amount of circulating pathogen before and after administering the composition. Inducing a therapeutic response may include reducing the degree or severity of at least one symptom of infection by the pathogen.

The compositions disclosed herein may be delivered via a variety of routes. Typical delivery routes include parenteral administration (e.g., intradermal, intramuscular, intraperitoneal, or subcutaneous delivery). Other routes include intranasal and intrapulmonary routes. Further routes include oral administration. Formulations of the pharmaceutical compositions may include liquids (e.g., solutions and emulsions), sprays, and aerosols.

The compositions disclosed herein may be co-administered or sequentially administered with other immunological, antigenic or vaccine or therapeutic compositions, including an adjuvant, or a chemical or biological agent given in combination with an antigen to enhance immunogenicity of the antigen.

Adjuvants

The compositions disclosed herein optionally include an adjuvant. The term “adjuvant” refers to a compound or mixture that enhances an immune response. An adjuvant can serve as a tissue depot that slowly releases the antigen and also as a lymphoid system activator that non-specifically enhances the immune response. Examples of adjuvants which may be utilized in the disclosed compositions include but are not limited to, co-polymer adjuvants (e.g., Pluronic L121® brand poloxamer 401, CRL1005, or a low molecular weight co-polymer adjuvant such as Polygen® adjuvant), poly (I:C), R-848 (a Th1-like adjuvant), resiquimod, imiquimod, PAM3CYS, aluminum phosphates (e.g., A1PO4), loxoribine, potentially useful human adjuvants such as BCG (Bacille Calmette-Guerin) and Corynebacterium parvum, CpG oligodeoxynucleotides (ODN), cholera toxin derived antigens (e.g., CTA1-DD), lipopolysaccharide adjuvants, complete Freund's adjuvant, incomplete Freund's adjuvant, saponin (e.g., Quil-A), mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

Prime-Boost Vaccination Regimen

As used herein, a “prime-boost vaccination regimen” refers to a regimen in which a subject is administered a first composition (e.g., a composition comprising one or more recombinant SARS-CoV-2 proteins as described herein), and then after a determined period of time (e.g., after about 2, 3, 4, 5, or 6 weeks), the subject is administered a second composition (e.g., a composition comprising one or more recombinant SARS-CoV-2 proteins as described herein), which may be the same or different than the first composition (e.g., different recombinant SARS-CoV-2 proteins, or combinations of proteins, may be included in the first and second compositions). The first composition (and the second composition) may be administered one or more times. The disclosed methods may include priming a subject with a first composition by administering the first composition at least one time, allowing a predetermined length of time to pass (e.g., at least about 2, 3, 4, 5, or 6 weeks), and then boosting by administering the same composition or a second, different composition.

For example, the methods may include administering a first pharmaceutical composition and optionally may include administering a second pharmaceutical composition to augment or boost an immunogenic response induced by the first pharmaceutical composition. The first and second pharmaceutical compositions may be the same or different. The optionally administered second pharmaceutical composition may be administered prior to, concurrently with, or after administering the first pharmaceutical composition. In some embodiments, the first composition is administered and then the second composition is administered after waiting at least about 4, 5, or 6 weeks. The first composition (and the second composition) may be administered one or more times.

Characterization of the Immune Response in Vaccinated Subjects

The pharmaceutical compositions disclosed herein may be delivered to subjects at risk for acquiring an infection by SARS-CoV-2. In order to assess the efficacy of an administered immunogenic composition or vaccine, the immune response can be assessed by measuring the induction of antibodies to particular epitopes of SARS-CoV-2 and/or cell-mediated responses against SARS-CoV-2. Antibody responses may be measured by assays known in the art such as ELISA. Titer or load of a pathogen may be measured using methods in the art including methods that detect nucleic acid of the pathogen. (See, e.g., U.S. Pat. No. 7,252,937, the content of which is incorporated by reference in its entirety). T-cell responses, also referred to as “cellular immune responses,” may be measured, for example, by using tetramer staining of fresh or cultured PBMC, ELISPOT assays or by using functional cytotoxicity assays, which are well-known to those of skill in the art. Immune responses also may be characterized by physiological responses. (See Li et al., Vaccine 28 (2010) 1598-1605; and Stemke-Hale et al., Vaccine 2005 Apr. 27; 23(23):3016-25, the content of which re incorporated herein by reference in their entireties.) Immune response also may be measured by pathological responses such as total weight loss or gain for the animal after challenge with SARS-CoV-2. Immune response also may be measured by pathological responses such as weight loss or gain for an organ of the animal after challenge with SARS-CoV-2.

Antigens and Dose

The compositions disclosed herein optionally may include an antigen, a panel of antigens, or a plurality of antigens. A “panel” or “plurality” or antigens as used herein means “more than one” and may mean more than 1, 2, 3, 4, 5, 10, 25, 50, or 100 antigens.

In some embodiments, the composition, kits, and methods contain or utilize a protein, polypeptide, peptide, or panel thereof as an antigen. The compositions, kits, and methods may be utilized to induce an antibody response and/or a cell-mediated response against infection by SARS-CoV-2.

Conventional vaccines and methods typically involve administering at least about 3 μg of an antigen per dose to a subject. (See, e.g., Scheifele et al. 2005, Hum. Vaccin. 1:180-186; Evans et al. 2001, Vaccine 19:2080-2091; and Kenney et al., N. Engl. J. Med. 351:2295-2301, the contents of which are incorporated herein by reference in their entireties). However, a dose as low as 1 μg of an antigen per dose to a subject also has been proposed. (See U.S. Pat. No. 6,372,223, the content of which is incorporated herein by reference in its entirety).

Suitable antigens may include polypeptides, peptides, or panels thereof that comprise one or more epitopes of a protein associated with a disease. For example, suitable polypeptides, peptides, or panels thereof may comprise one or more epitopes of a protein associated with a pathogen. Suitable polypeptides may comprise the full-length amino acid sequence of a corresponding protein of a pathogen or a fragment thereof. For example, suitable fragments may include 5-200 amino acids (or from 5-150, 5-100, 5-50, 5-25, 5-15, 10-200, 10-100, 10-50, 10-25, 10-25, or 10-15 amino acids) and include at least one epitope of the protein from which the fragment is derived. Suitable antigens for the compositions, kits, and methods may include panels of peptides derived from a protein of a pathogen. For example, a suitable antigen may comprise a panel of at least 2, 3, 4, 5, 10, 25, 50, 100, or more different peptides comprising at least about a 10-20 amino acid sequence from a protein of a pathogen. The different peptide antigens may overlap at the N-terminus, the C-terminus, or both termini with at least one other peptide antigen of the composition, for example, by at least about 1, 2, 3, 4, 5, 6, 7, 8, 9, or 10 amino acids.

Serological Assays

Serology tests are blood-based tests that can be used to identify whether a subject has been exposed to a particular pathogen. Serology-based tests typically analyze the serum component of whole blood. The serum includes antibodies to specific antigenic components of pathogens. These antigens are recognized by the immune system as foreign and are targeted by the immune response.

These types of tests are often used in viral infections to see if the patient has an immune response to a pathogen of interest, such as influenza. The tests can be used to diagnose infection. There are several types of serology tests, including but not limited to neutralization tests, chemiluminescent immunoassays (CLIA), enzyme-linked immunosorbent assays (ELISAs), and lateral flow assays (LFAs), also termed rapid diagnostic tests (RDTs).

Neutralization tests can indicate whether the patient has active, functional antibodies to the pathogen in question by measuring how much the patient antibodies can inhibit viral growth in the lab. This can be used with SARS-CoV-2 virus in a BSL-3 setting, or pseudoviruses that express certain SARS-CoV-2 proteins in a lower BSL setting.

Chemiluminescent immunoassay (CLIA) shows whether a patient has antibodies to a pathogen by displaying a fluorescent signal when patient antibodies interact with virus proteins.

Enzyme-linked immunosorbent assays (ELISAs) are more rapid serology tests performed in a lab that provide a readout of antigen-antibody interactions. Essentially, patient antibodies are “sandwiched” between the viral protein of interest and reporter antibodies, so that any active patient antibodies are detected.

Lateral flow assays (LFAs), also called rapid diagnostic tests (RDTs) display a colorimetric, qualitative readout of the presence of antibodies. These are often used in point-of-care settings. The patient sample is flowed over a membrane that has the target antigen anchored. If the sample contains antibodies specific to that antigen, they form a complex that results in a colored band on the strip.

The recombinant proteins disclosed herein are useful in serological testing, for example in CLIA, ELISA, and LFA formats. By way of example only, but not by way of limitation, in some embodiments, isolated recombinant proteins described above (with non-limiting examples shown in FIGS. 1-4 and 7-17), are covalently linked to the surface of fluorescent dye-labeled, magnetic microsphere beads. Serum antibodies are then captured by specific antigen-coupled beads, and captured antibodies are then bound by a labeled (e.g., fluorescently labeled, such as phycoerythrin, “PE”), secondary antibody recognizing conserved regions of immunoglobulin isotypes G, M, and A. When analyzed, for example, on the Luminex MagPix dual-laser cytometer, individual beads are identified by a fluorescent signature that indicates the presence of specific antibodies in the sample. In some embodiments, the recombinant antigen is linked to a plate (as for an ELISA assay), or is anchored to a membrane (as for lateral flow assays). Secondary, and tertiary antibodies can then be used to visualize a subject's antibody binding to the antigen.

This technology will help meet an urgent need for serological assays to track COVID-19 exposure and response in patients and the community.

A multiplexed, laboratory-developed assay utilizing custom made viral protein constructs provides a versatile platform for characterizing diverse, antigen-specific antibody responses in peripheral and respiratory tract specimens. This technology will enable a robust serological assay with high sensitivity and low background and that uses 1000× less protein antigens to effectively assay patient samples. The tagged protein constructs are optimized for binding efficiency and antigen display.

Methods of obtaining a subject sample comprising antibodies (e.g., obtaining a blood sample and preparing the sample for serological testing) are well known in the art.

Likewise, methods for conjugating, linking, or coupling a recombinant protein, such as those disclosed herein, to a solid support are well known in the art. By way of example, but not by way of limitation, such methods include absorption, trapping the protein within a gel matrix, and covalent linkage.

Exemplary Uses

Disclosed are recombinant proteins, compositions, vectors, kits, and methods for inducing an immune response against SARS-CoV-2, and/or for detecting whether a subject has been infected with SARS-CoV-2. The recombinant sequences disclosed herein are expected to be highly immunogenic, produced easily in bacterial as well as eukaryotic cells, and easily purified in large quantities. This technology will help meet an urgent need in the production of antibodies and diagnostics for COVID-19 treatment. Furthermore, these purified proteins can be used as immunogens to generate vaccines or neutralizing antibodies in small or large animals. This technology will enable reliable production and purification of antigens (protein sequences) that can be injected into animals to produce human polyclonal antibodies for SARS-CoV-2 infections. These antigens can also be used to measure humoral immune responses/cell mediated immune responses. In addition, the recombinant sequences disclosed herein are useful in serological assays to detect the presence of SARS-CoV-2 antibodies in a subject sample.

Thus, in some embodiments, the compositions, vectors, kits, and methods may be utilized to immunize humans against disease associated with SARS-CoV-2 infection such as COVID-19 and related complications.

In other embodiments, the compositions, vectors, kits, and methods may be used as antigens for the production of commercial-scale antibodies against SARS-CoV-2 virus. The antibodies may be administered for prophylactic or therapeutic purposes, e.g., to treat or prevent infection from SARS-CoV-2 in a subject in need thereof, and as a diagnostic tool to detect the presence of SARS-CoV-2 in a subject.

In further embodiments, the compositions, vectors, kits, and methods are used in serological assays to detect the presence of SARS-CoV-2 antibodies in a subject sample.

Kits of the present disclosure may include one or more of the following components: (a) an expression vector comprising a nucleic acid sequence encoding one or more recombinant SARS-CoV-2 proteins; (b) one or more isolated SARS-CoV-2 recombinant proteins; (c) cells comprising one or more expression vectors comprising the nucleic acid sequence encoding one or more recombinant SARS-CoV-2 proteins; (d) components useful to isolate or purify recombinant SARS-CoV-2 proteins expressed by a host cell; (e) instructions for use. In some embodiments, kits may include components for serological assays, such (a) one or more isolated SARS-CoV-2 recombinant proteins; and (b) and one or more labeled antibodies to detect binding of the subject antibodies to the SARS-CoV-2 recombinant proteins. In some embodiments, the isolated SARS-CoV-2 recombinant proteins is linked to a solid support, such as a bead, plate, slide, or membrane.

Methods of the present technology include administering compositions disclosed herein (including one or more recombinant SARS-CoV-2 proteins) to a subject in need thereof. In some embodiment, the composition is in the form of a vaccine.

Additional methods include administering the compositions disclosed herein (including one or more recombinant SARS-CoV-2 proteins) to one or more animals (e.g., avians, mammals) to induce an immune response. In some embodiments, the induced immune response results in the production of polyclonal antibodies that can be isolated from the immunized animal and used for therapeutic and diagnostic purposes. By way of example, but not by way of limitation, animals useful for antibody production include horses, cows, birds (e.g., chickens), mice, rats, rabbits, and goats.

### Exemplary Embodiments

The following exemplary embodiments are presented for illustrative purposes only and are not intended to, in any way, limit the scope of the instant disclosure.
1. A recombinant protein comprising: (i) a SARS-CoV-2 polypeptide sequence derived from the spike (“S”) protein amino acids 1-1220 or a variant thereof, and (ii) one or more heterologous polypeptide sequences selected from a purification tag, a detectable label, a flexible linker, a cleavage site to allow for tag removal after purification, a secretion signal peptide, and a solubility enhancer peptide.
2. The recombinant protein of embodiment 1, wherein the furin site “RRAR” in the polypeptide is genetically engineered so as not to be cleaved by furin, optionally wherein the furin site is engineered to “GSAS.”
3. The recombinant protein of embodiment 1, wherein the SARS-CoV-2 polypeptide sequence comprises a fragment of the S protein including amino acids 319-591, or a variant thereof.
4. The recombinant protein of any of the foregoing embodiments, wherein the heterologous polypeptide sequence comprises one or more of (a) a purification tag comprising a HIS tag; (b) a detectable label comprising Green Fluorescent Protein or enhanced Green Fluorescent Protein; (c) a flexible linker comprising GGGGSGGGGSGG (SEQ ID NO: 34); (d) a cleavage site to allow for tag removal after purification comprising a Tobacco Etch Virus nuclear-inclusion-a endopeptidase (TEV protease) recognition sequence, GENLYFQG (SEQ ID NO: 35); (e) a secretion signal peptide comprising MFLLTTKRT (SEQ ID NO: 36); and (f) a solubility enhancer peptide comprising maltose binding protein (MBP).
5. The recombinant protein of any of the foregoing embodiments, wherein the SARS-CoV-2 polypeptide sequence is selected from the group consisting of:
   (a) amino acids 1-1220 of the S protein, wherein the furin site is genetically engineered from RRAR (SEQ ID NO: 33) to GSAS (SEQ ID NO: 32);
      (b) amino acids 319-591 of the S protein; and
      (c) the N protein.
6. The recombinant protein of embodiment 7, wherein the heterologous protein is one or more selected from the group consisting of: (a) a purification tag comprising a HIS tag; (b) a detectable label comprising Green Fluorescent Protein or enhanced Green Fluorescent Protein; (c) a flexible linker comprising GGGGSGGGGSGG (SEQ ID NO: 34); (d) a cleavage site to allow for tag removal after purification comprising a Tobacco Etch Virus nuclear-inclusion-a endopeptidase (TEV protease) recognition sequence, GENLYFQG (SEQ ID NO: 35); (e) a secretion signal peptide comprising MFLLTTKRT (SEQ ID NO: 36); and (f) a solubility enhancer peptide comprising maltose binding protein (MBP).
7. The recombinant protein of any of the foregoing embodiments, wherein the SARS-CoV-2 polypeptide sequence is amino acids 1-1220 of the S protein, wherein the furin site is genetically engineered from RRAR (SEQ ID NO: 33) to GSAS (SEQ ID NO: 32); and wherein the heterologous protein consists of (a) TEV protease recognition sequence; (b) the flexible linker GGGGSGGGGSGG (SEQ ID NO: 34); (c) enhanced Green Fluorescent Protein; and (d) a 12×histag; and optionally wherein the recombinant protein forms a multimer.
8. The recombinant protein of any of the foregoing embodiments, wherein the SARS-CoV-2 polypeptide sequence is amino acids 319-591 of the S protein; and wherein the heterologous protein consists of (a) TEV protease recognition sequence; (b) the flexible linker GGGGSGGGGSGG (SEQ ID NO: 34); (c) enhanced Green Fluorescent Protein; and (d) a 12×histag; and optionally wherein the recombinant protein forms a multimer.
9. The recombinant protein of any of the foregoing embodiments, wherein the SARS-CoV-2 polypeptide sequence is amino acids 319-591 of the S protein; and wherein the heterologous protein consists of (a) TEV protease recognition sequence; (b) the flexible linker GGGGSGGGGSGG (SEQ ID NO: 34); (c) enhanced Green Fluorescent Protein; and (d) a 10×histag; and optionally wherein the recombinant protein forms a multimer.
10. The recombinant protein of any of the foregoing embodiments, wherein the SARS-CoV-2 polypeptide sequence is the S protein; and wherein the heterologous protein consists of (a) TEV protease recognition sequence; (b) the flexible linker GGGGSGGGGSGG (SEQ ID NO: 34); (c) enhanced Green Fluorescent Protein; (d) a 12×histag; and (e) MBP; and optionally wherein the recombinant protein forms a multimer.
11. The recombinant protein of any of the foregoing embodiments, comprising a secretion signal for insect cells.
12. The recombinant protein of embodiment 11, wherein the secretion signal comprises MFLLTTKRT (SEQ ID NO: 36).
13. A recombinant protein comprising: (i) one or more viral polypeptide sequences, and (ii) one or more heterologous polypeptide sequences,
   wherein the viral polypeptide sequences comprises a SARS-CoV-2 polypeptide sequence derived from one or more of the nucleocapsid (“N”) protein or a variant thereof, the envelope (“E”) protein or variant thereof, or the membrane (“M”) protein or a variant thereof; and
   wherein the heterologous polypeptide sequence comprises one or more of a purification tag, a detectable label, a flexible linker, a cleavage site to allow for tag removal after purification, a secretion signal peptide, and a solubility enhancer peptide.
14. The recombinant protein of embodiment 13, wherein the SARS-CoV-2 polypeptide sequence comprises the N protein or a variant thereof.
15. The recombinant protein of any of the foregoing embodiments, comprising the polypeptide sequence of SEQ ID NO: 7.
16. The recombinant protein of any of the foregoing embodiments, comprising the polypeptide sequence of SEQ ID NO: 8.
17. The recombinant protein of any of the foregoing embodiments, comprising the polypeptide sequence of SEQ ID NO: 9.
18. The recombinant protein of any of the foregoing embodiments, comprising the polypeptide sequence of SEQ ID NO: 10.
19. The recombinant protein of any of the foregoing embodiments, comprising the polypeptide sequence of SEQ ID NO: 11.
20. The recombinant protein of any of the foregoing embodiments, comprising the polypeptide sequence of SEQ ID NO: 12.
21. The recombinant protein of any of the foregoing embodiments, comprising the polypeptide sequence of SEQ ID NO: 13.
22. An expression vector, comprising a nucleic acid sequence encoding the recombinant protein of any of the foregoing embodiments.
23. A cell comprising the expression vector of embodiment 15.
24. A pharmaceutical composition comprising the recombinant protein of any of embodiments 1-21.
25. The pharmaceutical composition of embodiment 24 formulated as a vaccine.
26. A method of inducing an immune response against SARS-CoV-2 in a subject in need thereof, the method comprising administering to the subject an effective amount of the vaccine of embodiment 25 to induce an immune response against SARS-CoV-2 in the subject.
27. The recombinant protein of any of embodiments 1-21, linked to a solid support.
28. The recombinant protein of embodiment 27, wherein the solid support is selected from the group consisting of a bead, a plate, a slide, and a membrane.
29. A method for identifying whether a subject has been exposed to SARS-CoV-2, the method comprising:
   (a) obtaining a sample from the subject;
   (b) contacting the sample with the recombinant protein of any of embodiments 1-21 under conditions that allow SARS-CoV-2 antibodies, if present in the sample, to bind to the recombinant protein and form an antibody-antigen complex; and
   (c) detecting the complex.
30. The method of embodiment 29, wherein the complex is detected by contacting the complex with a secondary antibody that binds the complex and comprises a detectable label, optionally wherein the secondary antibody is an anti-human antibody that binds human SARS-CoV-2 antibodies and comprises a fluorometric label or colorimetric label.
31. A recombinant protein comprising the amino acid sequence SEQ ID NO: 18.
32. A recombinant protein comprising the amino acid sequence SEQ ID NO: 19.
33. A recombinant protein comprising the amino acid sequence SEQ ID NO: 20.
34. A recombinant protein comprising the amino acid sequence SEQ ID NO: 21.
35. A recombinant protein comprising the amino acid sequence SEQ ID NO: 22.
36. A recombinant protein comprising the amino acid sequence SEQ ID NO: 23.
37. A recombinant protein comprising the amino acid sequence SEQ ID NO: 24.
38. A recombinant protein comprising the amino acid sequence SEQ ID NO: 25.
39. A recombinant protein comprising the amino acid sequence SEQ ID NO: 26.
40. A recombinant protein comprising the amino acid sequence SEQ ID NO: 27.
41. A recombinant protein comprising the amino acid sequence SEQ ID NO: 28.
42. A recombinant protein comprising the amino acid sequence SEQ ID NO: 29.
43. A recombinant protein comprising the amino acid sequence SEQ ID NO: 30.
44. A recombinant protein comprising the amino acid sequence SEQ ID NO: 31.
45. An expression vector, comprising a nucleic acid sequence encoding the recombinant protein of any one of embodiments 31-44.
46. A cell comprising the expression vector of embodiment 45.
47. The recombinant protein of any of embodiments 31-44, linked to a solid support.
48. The recombinant protein of embodiment 47, wherein the solid support is selected from the group consisting of a bead, a plate, a slide, and a membrane.
49. A method for identifying whether a subject has been exposed to SARS-CoV-2, the method comprising:
   (a) obtaining a sample from the subject;
   (b) contacting the sample with the recombinant protein of any of embodiments 31-42 under conditions that allow SARS-CoV-2 antibodies, if present in the sample, to bind to the recombinant protein and form an antibody-antigen complex; and
   (c) detecting the complex.
48. The method of embodiment 49, wherein the complex is detected by contacting the complex with a secondary antibody that binds the complex and comprises a detectable label, optionally wherein the secondary antibody is an anti-human antibody that binds human SARS-CoV-2 antibodies and comprises a fluorometric label or colorimetric label.

### Examples

The following examples are illustrative and are not intended to limit the disclosed subject matter.

### Example 1: SARS-CoV-2 Antigen Design, Production, and Testing

Bioinformatic analysis of SARS-CoV-2 genome sequences to identify diverse and most immunogenic sequences for the generation of a robust immune response. Bioinformatic analyses were carried out as follows. Over 200 full-length sequences of SARS-CoV-2 genomes were downloaded from GISAID (gisaid.org) and their gene coding regions were annotated using VGAS (Viral Genome Annotation System). Four major structural proteins including S-spike surface glycoprotein; E-small envelope protein; M-matrix protein; and N-nucleocapsid protein were investigated to identify a diverse set of antigens that could elicit robust immune responses. Specifically, the receptor binding domain (RBD) of spike protein is considered highly immunogenic, hence both RBD and the ecto-domain regions of S have been major targets of neutralizing antibodies in other betacoronavirus outbreaks such as MERS and SARS. RBD binds to the human ACE2 (Angiotensin Converting Enzyme 2) receptor to gain entry into the host cells. Multiple sequence alignments were carried out followed by the identification of consensus sequences for each structural protein. Homology models were developed for different variants of proteins using Schrodinger [schrodinger.com] and I-TASSER software to investigate specific binding regions between viral and host proteins. Epitope mapping is in progress on the structural proteins using different length peptides (8-15 amino acids) and different HLA alleles using NetMHCcons software to determine the MHC-peptide binding affinities and rank the most diverse and immunogenic epitopes. At this time the RBD and the ecto-domain sequences of the S protein and the N protein were advanced to cloning, protein expression and testing. Expression vectors for S, E, M and N proteins were also created for mammalian cell culture.

Cloning, production, purification of antigens. This research is ongoing as we optimize our SARS-CoV-2 protein purifications and create new versions of the expression constructs. The amino acid sequence used in each expression construct were designed based on the bioinformatics analysis. Below, four S and two N protein constructs are described.

Three expression constructs for S protein expression in insect cells and one for N protein employing the pVL1392 and pVL1392 plasmid are described below. To stabilize the Spike protein Ecto domain the furin site (RRAR) was mutated to GSAS. Codons were optimized for bacterial, insect cell, or mammalian cell culture, as appropriate. A TEV protease recognition sequence (GENLYFQG (SEQ ID NO: 35)) was inserted for tag removal after purification. TEV is followed by a flexible linker (GGGGSGGGGSGG (SEQ ID NO: 34)) and then enhanced green fluorescent protein (eGFP) is included to ease tracking the expressed protein during purification. A 12×His tag is at the C-terminus for nickel affinity purification. This C-terminal tag system is used with the receptor binding domain (RBD) and N protein expression constructs.

Insect Cell Expression Constructs:

(1) “S-Ecto(1-1220)GSAS”-TEV-linker-eGFP-12×Histag (see FIG. 2)

This expression vector includes the furin mutation and an insect cell secretion signal peptide at the N-terminus.

(2) “S-RBD(319-591)”-TEV-linker-eGFP-12×Histag (see FIG. 1).

There are two forms of this expression vector that we are currently testing to see which gives active protein and highest yield. One form is cytoplasmically expressed and the other is secreted.

(3) N-TEV-linker-eGFP-12×Histag (see FIG. 3).

There are two forms of this expression vector that we are currently testing to see which gives active protein and highest yield. One form is cytoplasmically expressed and the other is secreted.

A bacterially expressed construct that appears to be successful for the spike RBD is a pET28a construct that includes a 10×His tag on the N-terminus, followed by maltose binding protein to improve solubility, the flexible linker, the TEV protease site and then the S-RBD at the C-terminus. For proper folding and correct disulfide bond formation the RBD plasmid was coexpressed with the CyDisCo system.

Bacterial Expression Construct:

(4) 10×-His-MBP-linker-TEV—S-RBD(319-591) (see FIG. 4).

This bacterially expressed fusion protein can be purified via nickel and/or amylose affinity chromatography.

### Example 2: SARS—Co-V-2 Antigen Design for Serological Assays

We next used the viral protein sequences and expression constructs in Example 1 to serve as antigens in serological assays. We performed Luminex assays with COVID-19 positive and negative serum samples. Three of the antigens could differentiate between the two patient groups with different levels of sensitivity.

Cloning, Production, Purification of Antigens:

This research is ongoing as we optimize our SARS-CoV-2 protein purifications and create new versions of the expression constructs. The next version of these proteins will further explore the utility of MBP fusions and will test the incorporation of a polylysine repeating sequence on the termini to further direct chemical linking to the Luminex microspheres and optimize antigen presentation. Below the four S and two N protein constructs we are currently moving forward with are described.

Three expression constructs for S protein expression in insect cells and one for N protein employing the pVL1392 plasmid are described below. To stabilize the Spike protein Ecto domain the furin site (RRAR (SEQ ID NO: 33)) was mutated to GSAS (SEQ ID NO: 32). Codons were optimized for bacterial, insect cell or mammalian cell culture, as appropriate. A TEV protease recognition sequence (GENLYFQG (SEQ ID NO: 35)) was inserted for tag removal after purification. TEV is followed by a flexible linker (GGGGSGGGGSGG (SEQ ID NO: 34)) and then enhanced green fluorescent protein (eGFP) is included to ease tracking the expressed protein during purification. A 12×His tag is at the C-terminus for nickel affinity purification. This C-terminal tag system is used with the receptor binding domain (RBD) and N protein expression constructs.

Insect Cell Expression Constructs:

(1) “S-Ecto(1-1220)GSAS”-TEV-linker-eGFP-12×Histag (see FIG. 2).

This expression vector includes the furin mutation and an insect cell secretion signal peptide at the N-terminus.

(2) “S-RBD(319-591)”-TEV-linker-eGFP-12×Histag (see FIG. 1)

There are two forms of this expression vector that we are currently testing to see which gives active protein and highest yield. One form is cytoplasmically expressed and the other is secreted.

(3) N-TEV-linker-eGFP-12×Histag (see FIG. 3)

There are two forms of this expression vector that we are currently testing to see which gives active protein and highest yield. One form is cytoplasmically expressed and the other is secreted.

One bacterially expressed construct appears to be successful for the spike RBD. This pET28a construct includes a 10×His tag on the N-terminus, followed by maltose binding protein to improve solubility, the flexible linker, the TEV protease site and then the S-RBD at the C-terminus. For proper folding and correct disulfide bond formation the RBD plasmid is coexpressed with the CyDisCo system.

Bacterial Expression Construct:

(4) 10×-His-MBP-linker-TEV—S-RBD(319-591) (see FIG. 4).

This bacterially expressed fusion protein can be purified via nickel and/or amylose affinity chromatography.

Coupling to Luminex beads. The purified protein antigens described above are covalently linked to the surface of fluorescent dye-labeled, magnetic microsphere beads. Serum antibodies are captured by specific antigen-coupled beads, and captured antibodies are then bound by a PE-labeled secondary antibody recognizing conserved regions of immunoglobulin isotypes G, M, and A. When analyzed on the Luminex MagPix dual-laser cytometer, individual beads are identified by a fluorescent signature that indicates the presence of specific antibodies in the sample.

Preliminary assay with patient samples. A validation study performed on a preliminary configuration of the serology assay using the antigen constructs described here successfully demonstrated sensitivity and specificity for SARS-CoV-2 antibodies in serum and plasma.

### Example 3: SARS-CoV-2 Antigen Design for Serological Assays

Additional sequences, specific for COVID serology antibody detection and which can differentiate SARS-CoV-2 variants, were also developed using methods similar to the methods described above in Examples 1 and 2 (data not shown).

These sequences are shown in FIGS. 7-17.

This technology will help meet an urgent need for serological assays to track COVID-19 exposure and response in patients and the community.

A multiplexed, laboratory-developed assay utilizing custom made viral protein constructs provides a versatile platform for characterizing diverse, antigen-specific antibody responses in peripheral and respiratory tract specimens. This technology will enable a robust serological assay with high sensitivity and low background and uses 1000× less protein antigens to effectively assay patient samples. The tagged protein constructs are optimized for binding efficiency and antigen display.

### Example 4: Insect Cell Expression and Purification of Recombinant SARS-COV-2 Spike Proteins that Demonstrate ACE2 Binding

Although existing previously, coronaviruses have only had their descriptive name (meaning “crown”) since 1962.¹ With a total of 24 similar species in the wild, only seven are known to cause disease in humans.²⁻⁶ Of these seven, the most pathogenic members are severe acute respiratory syndrome coronavirus (SARS-CoV, originating in Guangdong China in 2002), Middle East Respiratory Syndrome coronavirus (MERS-CoV, originating in the Arabian Peninsula in 2012), and the novel coronavirus that emerged in December of 2019 in Wuhan, China.^{4, 7} In January of 2020, this new coronavirus was named SARS-CoV-2 as the causative agent of the disease, COVID-19. It is most closely related to SARS-CoV with a genomic sequence identity of 79%.^{8, 9} On Mar. 11, 2020, a COVID-19 global pandemic was declared by the WHO.¹⁰ As of Feb. 25, 2022, there are 5,911,081 confirmed deaths worldwide due to COVID-19 (https://covid19.who.int/). The progression of the pandemic saw variants of SARS-CoV-2 emerge, some with increased virus transmission resulting in a global spread.¹¹⁻¹⁵ Currently, the omicron variant (https://www.who.int/en/activities/tracking-SARS-CoV-2-variants/) is responsible for the majority of the infections in the United States (https://covid.cdc.gov/covid-data-tracker/#variant-proportions). The omicron variant shows the potential to be not only more infectious than the delta variant with a higher chance of reinfection amongst previously infected individuals, but can significantly evade immunity achieved through current vaccinations.¹⁶⁻¹⁹ Recent emergence of the fast-spreading Omicron variant has re-invigorated efforts to develop more effective countermeasures against COVID-19.¹⁸ Effective vaccines are available to fight the spread of the disease, but each new variant carries the possibility to evade the immunity offered by existing vaccines. Research into SARS-CoV-2 remains vital to curtail the pandemic.

Coronaviruses are positive sense, single-strand RNA viruses with four structural proteins: spike (S), envelope (E), membrane (M), and nucleocapsid (N).^{8, 20, 21} The S protein forms a homo-trimer with each protein composed of two subunits named 51 (residues 14-685) and S2 (residues 686-1273).^{9, 22, 23} is S1 responsible for the initial binding to the aminopeptidase N segment of the angiotensin-converting enzyme (ACE2) receptor, whereupon it is shed, and the S2 subunit mediates fusion with the target cell membrane.^{22, 24} This interaction between the S protein and the ACE2 receptor of the host's cells is required for virus entry into the cell. Binding occurs with low nanomolar affinity.^{9, 22} The S protein is highly glycosylated, allowing it to evade the host organism's immune system by using glycosylation to mask up to 40% of the S protein's surface area.^{23, 25, 26} Accounting for this potential epitope shielding by glycosylation makes expressing S protein in an expression system that will include such glycosylation of spike protein far more useful for immunological assays.

It has recently been shown that transcription of ACE2 is regulated by bromodomain and extraterminal domain (BET) proteins that bind acetylated residues on histones to recruit transcriptional machinery.^{27, 28} Inhibitors of BET proteins have been shown to decrease ACE2 expression, spike protein binding, and SARS-CoV-2 infection in lung epithelial cells and cardiomyocytes.^{29, 30} Anti-ACE2 antibodies can similarly attenuate viral entry and infection through the occupation of ACE2 receptors, thus blocking spike protein binding (i.e., ACE2 receptor blockade).²⁷ The SARS-CoV-2 envelope protein is additionally known to directly interact with BET proteins, but no associated effects have been reported for viral infection.²⁸

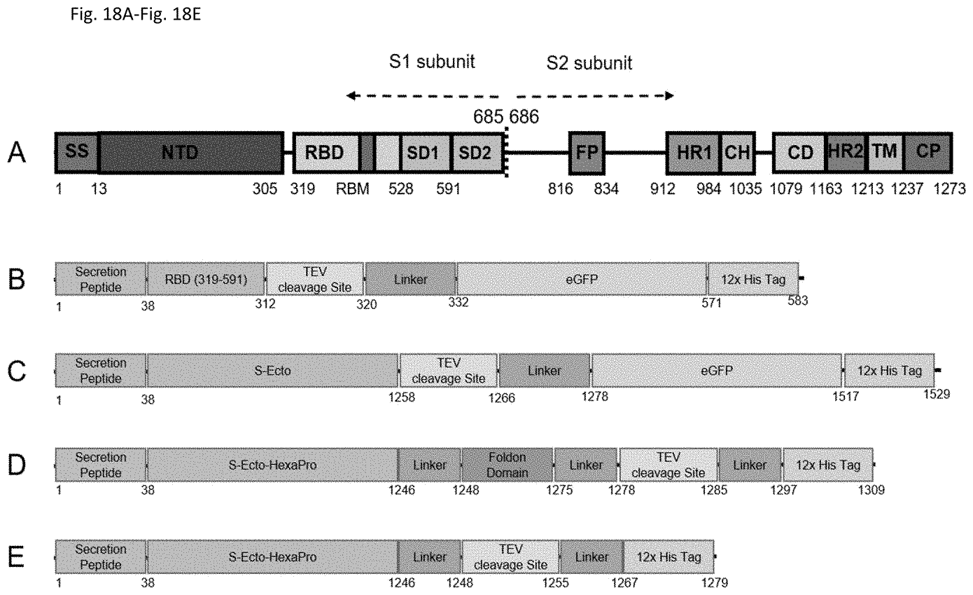

With the shift in focus of many laboratories toward COVID-19 research, an efficient and reliable method for purifying large amounts of glycosylated S protein is needed. We chose to use baculovirus-based recombinant spike protein expression in insect cells which provide N-glycosylation that mirrors mammalian cells, albeit with simpler side chains with terminal mannose residues instead of sialic acid residues.³¹ Insect cell expression systems offer significant advantages over mammalian cell line expression systems. The proteins from insect cell expression systems can have complex post-translational modifications and show immunogenicity, antigenicity, and biological activity similar to authentic natural proteins. Additional benefits include less expensive media, ease of scalability, vectors that are safe for humans, and a greatly reduced turn-around time from starting a culture to expressing protein.³² In this disclosure, we demonstrate the expression of four constructs of the original SARS-CoV-2 S protein ectodomain in insect cells using recombinant baculovirus and purification of each using a simple and robust method (FIG. 18).^{21, 24} The spike protein receptor binding domain (RBD) enhanced green fluorescent protein (eGFP) fusion construct (S-RBD-eGFP) was designed to be useful for biological studies. It is comprised of the RBD portion of the S protein that includes the SD1 domain (319-591) with eGFP and 12× His tag bound by a TEV cleavable linker domain on the C-terminus. The linked eGFP makes the protein easy to track during purification and gives confidence that the construct is folded correctly, as there is a strong relationship between the correct folding of the C-terminal eGFP chromophore and the absence of the upstream linked protein forming inclusion bodies or aggregating.^{33, 34} The S-Ecto-eGFP construct is much the same, but with the full-length spike ectodomain replacing the RBD. The S-Ecto-HexaPro(+F) construct is nearly identical to one previously expressed and tested with mammalian expiCHO cells.³⁵ This construct contains six proline mutations that stabilize the protein, a C-terminal foldon domain to assist with trimerization, and a TEV cleavable 12× His tag attached by a flexible linker region.^{36, 37} This construct was included in this study to test if the proline mutations will increase protein production in insect cells. The S-Ecto-HexaPro(−F) construct is the same as the S-Ecto-HexaPro(+F) construct, only with the foldon domain removed. This was done to test if the foldon domain has an impact on protein expression levels. All four of the constructs tested had an insect cell secretion peptide added to the N-terminus, and the three ectodomain constructs had the furin cleavage site eliminated (682-685 on S-Ecto-eGFP) by mutation from RRAR to GSAS, preventing S1/S2 subunit cleavage.^{4, 37}

Each of the purified constructs was tested for binding activity using surface plasmon resonance (SPR), demonstrating that the proteins contain active and functionally folded RBD by their ability to bind ACE2. In addition, we show that the ACE2 receptor blockade or treatment with BET inhibitors (e.g. JQ1 and RVX-208) significantly reduced the binding between the spike protein and ACE2 in cultured human epithelial cells.

Methods

Virus Creation

The four spike protein sequences were ordered from GenScript in pET28a vectors: S-Ecto-HexaPro(+F), S-Ecto-HexaPro(−F), S-RBD-eGFP, and the S-Ecto-eGFP (S1). SF9 cells and BestBac™ 2.0 linearized Baculovirus DNA were acquired from Expression Systems (catalog number 91-200). Using the manufacturer's instructions and the aforementioned plasmids, P0, P1, and (if needed) P2 Baculovirus containing our sequence of interest were created.

Protein Expression

SF9 (Spodoptera frugiperda) and Tni (Trichoplusia ni) cells (Expression Systems) were grown in sterile PC flasks (Fisher Scientific) using ESF-921 media (Expression Systems), in a shaking incubator with a 2-inch orbit running at 160 RPM at 28° C. The day before infection, the cells were passaged to 1×10⁶ cells/ml into ESF-AF media (Expression Systems). When the cells reached 2×10⁶ cells/ml the following day, they were infected with the appropriate virus at an MOI of 5 and placed in a separate shaking incubator using the same conditions, whereupon they were left to shake for 72 hours. From this point on, all steps were performed at room temperature or higher to prevent misfolding of the protein.³⁸ After 72 hours, one cOmplete ULTRA protease inhibitor tablet (Roche) was dissolved in each liter of media, and the contents of the flasks were centrifuged at 400×g for 10 minutes. The supernatant was decanted and centrifuged a second time at 14,000×g for 30 minutes. The supernatant was then passed through a sterile filter and stored on the benchtop at room temperature in sterile conditions.

Protein Purification

All purification steps are performed on the bench at room temperature. INDIGO-Ni agarose resin (Cube Biotech) was rinsed with wash buffer (20 mM Tris, pH 8.0, 1 M NaCl), added to the media, and stirred at room temperature for three hours. After stirring, the resin was allowed to settle for 30 minutes. The media was then decanted and disposed of, and the resin suspended in wash buffer. The suspended resin was added to 30 ml gravity-flow columns (BIO-RAD) and washed with 10 CV of wash buffer. The column was washed with 3 CV of wash buffer containing 20 mM, 40 mM, 60 mM, 80 mM, and 100 mM imidazole were performed sequentially. The imidazole stock solution (1 M) was adjusted to pH 8 with NaOH before use. The protein was eluted with wash buffer containing 500 mM imidazole. Washes and elutions were concentrated using an AMICON® Ultra-15 100 kDa MWCO centrifugal concentrator (regenerated cellulose membrane, Millipore Sigma) and run on ExpressPlus™ PAGE gels (GenScript) and stained with SimplyBlue™ SafeStain (Novex) to confirm the location of the eluted protein. Protein concentration was determined by Amo using calculated extinction coefficients based on the amino acid sequence.
12×-His tag removal

Once the fraction with the correct molecular weight (MW) band had been identified, it was incubated with TEV protease[BG1] (1 mg of TEV protease for every 10 mg of target protein) either on the benchtop overnight or for 2 hours at 37° C. Once cleavage was complete, the protein was buffer exchanged into the wash buffer, and the sample was incubated with the same amount of INDIGO resin as used in the previous step, gently stirring on the benchtop for three hours. The mixture was then added to a 30 ml gravity-flow column (BIO-RAD), the column was washed with 3 CV of wash buffer, and the flow-through of this wash was collected. The cleaved 12×-His tags, His-TEV protease, and protein with uncleaved 12×-His tags were eluted from the column with 3 CV of wash buffer with added 500 mM imidazole. Amicon® Ultra 15 (Millipore) with a MWCO of either 30 kDa or 100 kDa were used to concentrate samples. The cleaved and uncleaved samples were run on SDS-PAGE to monitor cleavage success. The purity of the 12×-His tag cleaved fraction was confirmed by SDS-PAGE where 5 μg of the protein was loaded onto the gel.

Deglycosylation of the SARS-CoV-2 S-Ectodomain

Removal of N-glycans, 0-glycans, and Sialic acids from the purified S-Ectodomain was performed by using a Deglycosylation kit (Agilent, Calif., USA) as per the manufacturer instructions. Briefly, one μg of S-Ectodomain was used for N-glycanase, 0-glycanase, and Sialidase A (1 μl/reaction) treatment at 37° C. for 3 h. After treatment, the protein-enzyme mixtures were mixed with 2× Laemmli buffer and boiled at 100° C. for 5 min. The mixtures were resolved on 4-20% SDS-PAGE and blotted with PVDF membrane. The membrane was immunoprobed with an anti-Spike protein antibody as described.³⁹

Surface Plasmon Resonance (SPR) Based hACE2 Binding Assay

A sensor chip was captured with recombinant hACE2-AviTag protein from 293T cells (Acro Biosystems, DE, USA) followed by injection of 300 μl of freshly prepared serial dilutions of S-Ecto-HexaPro(+F), S-Ecto-HexaPro(−F), S-RBD-eGFP, and S-Ecto-eGFP made with Tni cells (Expression Systems) and passed over it at a flow rate of 50 μl/min (contact duration 180 seconds) for the association, and disassociation was performed over a 600-second interval. A mock surface and buffer-only injections were used to correct for background signal. Bio-Rad ProteOn Manager (version 3.1) was used for data processing.

Chemical Compounds

Apabetalone (RVX-208) was a kind gift from Resverlogix Corp. (Edmonton, Canada) and JQ1 was purchased from Cayman Chemicals. Both compounds were dissolved in dimethyl sulfoxide (DMSO).

Cell Culture

Human bronchial epithelial Calu-3 cells (a kind gift from Dr. Dickinson, UNMC; originally from ATCC) were maintained at 37° C. in a humidified environment enriched with 5% CO2 in Eagle's Minimum Essential Medium (EMEM, ATCC) supplemented with 10% fetal bovine serum (FBS), 100 U/mL penicillin, and 100 μg/mL streptomycin.

Spike Protein Binding

Calu-3 were seeded at 150,000 cells per well in 24-well plates and allowed to adhere for 24 h before treatment. Cells were treated with BET inhibitors (5 μM JQ1 or 10-20 μM RVX-208) or vehicle (DMSO) in complete growth medium for 24 h. For antibody blocking conditions, cells were incubated with 40 μg/mL anti-ACE2 (R&D Systems #AF933) or goat IgG isotype control (R&D AB108C) in PBS/2% FBS for 45 min, rocking at room temperature (RT). For staining, all test samples were incubated with 50 μg/mL S-Ecto-eGFP or control GFP-His tag protein (Sino Biological) and Zombie NIR™ Fixable Viability dye (BioLegend) in PBS/2% FBS for 30 min, rocking at RT. Samples were suspended in PBS/2% FBS for flow cytometry analyses.

Flow Cytometry

S-Ecto-eGFP protein binding to live Calu3 cells was measured using a NovoCyte 2060R flow cytometer and analyzed with NovoExpress version 1.4.1 software (Agilent Technologies).

Statistical Analysis

Statistical significance was determined through student's t-tests using GraphPad Prism v9 software (San Diego, Calif.). Comparisons were done versus isotype or vehicle control, and P<0.05 were considered statistically significant.

Results and Discussion

Purification of Spike-Ectodomain

The purification of S-Ecto-HexaPro(+F), S-Ecto-HexaPro(−F), S-RBD-eGFP, and S-Ecto-eGFP sequences all follow the same purification protocol. The affinity tag and optional eGFP domain are attached to the S-Ecto and S-RBD domains by a flexible linker that can be cleaved with TEV protease. This cleavage method was chosen because the remaining portion of the TEV protease cleavage sequence (ENLYQ (SEQ ID NO: 37)) is not found in the human proteome, and so would not produce a self-recognition epitope in humans. To ensure that the proteins are glycosylated, have high levels of expression, and that the cell culturing was as user-friendly as possible, we chose to express the protein in insect cells using a baculovirus vector. Sf9 and Tni cells were selected for this purpose as Sf9 cells are one of the most widely used for insect cell cultures, while Tni cells have been shown to express and secrete more recombinant protein than other insect cell lines. The MOI of 5 and incubation time of three days was chosen because it resulted in the optimal amount of protein when the media was tested by western blot (not shown).

We tested three types of immobilized metal affinity chromatography (IMAC) resins: Ni-NTA Agarose (Qiagen), Ni Sepharose excel (Cytiva), INDIGO-Ni agarose resin. We found that the cell media leached the nickel ions from the Ni-NTA Agarose resin, significantly reducing the final protein yield. Excel and INDIGO-Ni are both chemical resistant resins, and both showed no observable metal ion leaching. We decided to proceed with our experiments using the INDIGO-Ni resin, as the Excel resin co-purified more impurities (not shown).

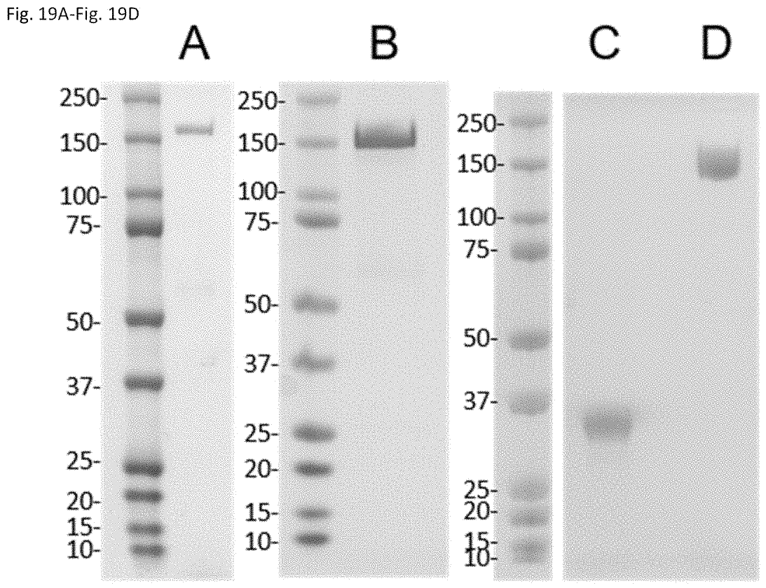

We took note of the differences in total protein purified for each construct and each cell line tested (Table 1). The protein yield from 1 L of cultured SF9 cells was 3.5 mg, 0.16 mg, 1.2 mg, and 0.58 mg for S-RBD-eGFP, S-Ecto-eGFP, S-Ecto-HexaPro(+F), and S-Ecto-HexaPro(−F), respectively. The yield from 1 L of cultured Tni cells was 0.2, 4.4, and 1 mg from S-Ecto-eGFP, S-Ecto-HexaPro(+F), and S-Ecto-HexaPro(−F), respectively. S-RBD-eGFP protein yield for Tni cells was not performed. After removal of the affinity tag (or affinity tag+eGFP), five μg of each protein was loaded onto SDS-PAGE and stained with SimplyBlue™ SafeStain. We see all the proteins run at higher molecular weight than what the expected protein size, presumably due to the presence of glycosylation. With the tags removed, S-RBD-eGFP has a calculated MW of 31.6 kDa and runs on SDS-PAGE at 36 kDa, S-Ecto-eGFP has a calculated MW of 136.0 kDa and runs at 160 kDa, S-Ecto-HexaPro(+F) has a calculated MW of 137.9 kDa and runs at 170.0 kDa, and the S-EctoHexaPro(−F) has a calculated MW of 134.6 kDa and runs at 170.0 kDa. The affinity tag (or affinity tag+eGFP) was cleaved from all constructs by TEV protease with varying efficiencies. Using the same type of resin binds the His-tagged TEV protease, cleaved His-tags, spike proteins with intact His-tags, and the non-specifically bound proteins eluted during the first step of purification. This results in extremely pure protein in the flow-through (FIG. 19). As tag removal would be required for certain experiments, it is important to be able to cleave the affinity tag from all three subunits of the spike ectodomain as an uncleaved tag on a single subunit will result in all three subunits being removed by the final nickel affinity column and the final protein yield will be reduced.

Table 1. Protein yield data. A comparison between yields of recombinant protein purified from 1 L of cell media after purification with a single round of INDIGO Ni agarose resin from both Sf9 cells and Tni cells, as well as the amount of pure protein that could be purified after tag cleavage with TEV protease. The calculated MW values are given alongside the observed MW values for the proteins with tags cleaved. S-RBD-eGFP protein yield in Tni cells was not performed.

| | Protein yield | Protein yield | | |
|---|---|---|---|---|
| | from Sf9 cells | from Tni cells | Calculated MW | Observed MW |
| Construct | (mg) | (mg) | (kDa) | (kDa) |
| S-RBD-eGFP | 3.5 | NA | 31.6 | 36.0 |
| (#186) | | | | |
| S-Ecto-eGFP | 0.16 | 0.2 | 136.0 | 160.0 |
| (#192) | | | | |
| S-Ecto- | 1.2 | 4.4 | 137.9 | 170.0 |
| HexaPro(+F) | | | | |
| (#314) | | | | |
| S-Ecto-HexaPro(−F) | 0.58 | 1 | 134.6 | 170.0 |
| (#326) | | | | |

We tested two types of centrifugal concentrator membranes for their effectiveness in concentrating these proteins: polyethersulfone and regenerated cellulose. We found that all three of the spike ectodomain proteins would bind in a non-recoverable fashion to the polyethersulfone membranes while the S-RBD-eGFP behaved normally. All the proteins tested behaved normally when concentrated with a regenerated cellulose membrane such as the Amicon® Ultra 15 (Millipore).

Deglycosylation of the SARS-CoV-2 S-Ecto-eGFP

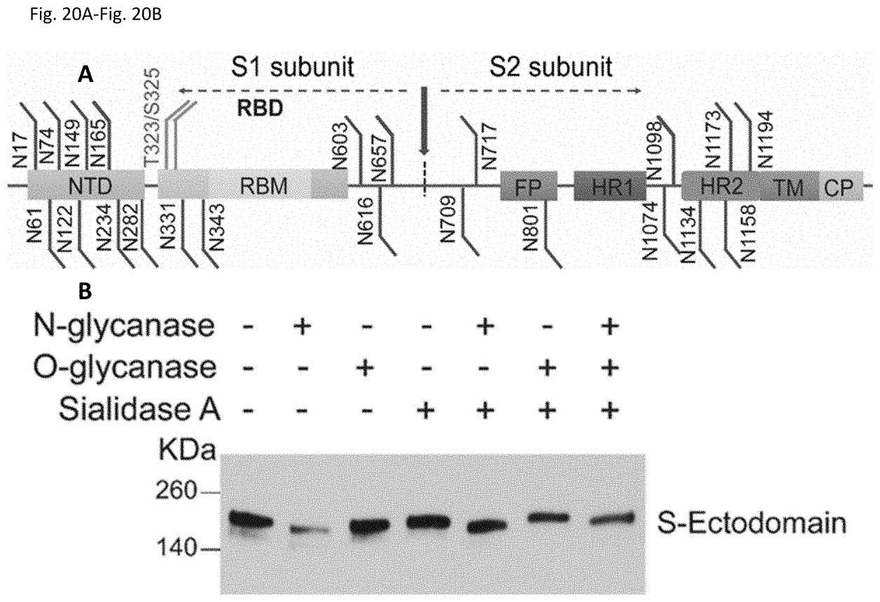

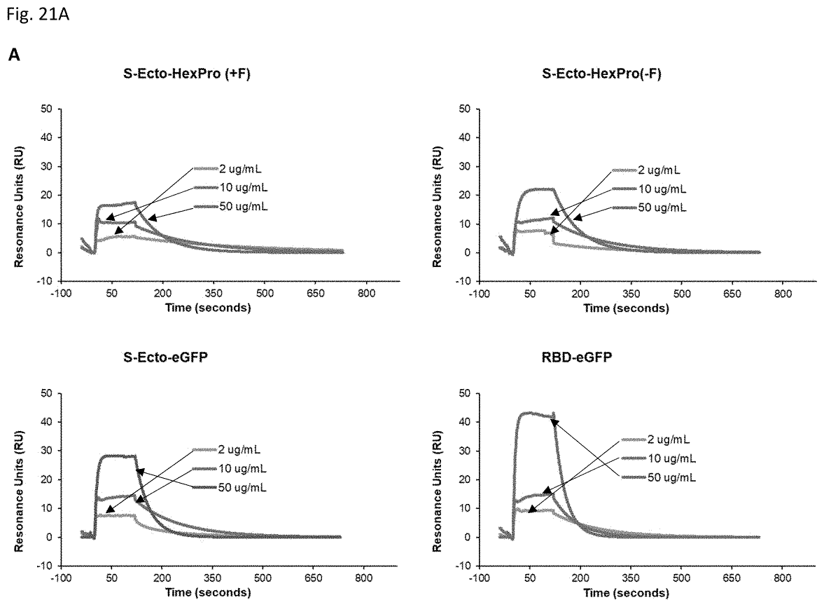

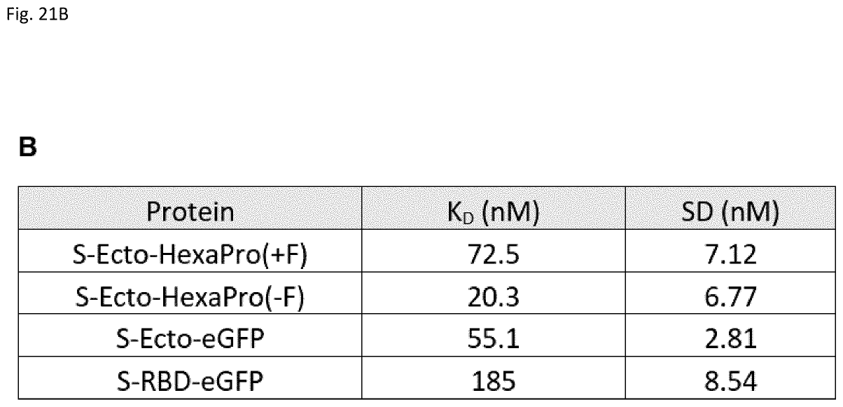

Spike ectodomain normally has 22 N-glycosylation and 2 O-glycosylation sites (FIG. 20A).^{25, 40, 41} We explored the type of glycosylation on the purified S-Ecto-eGFP by treatment with N-glycanase (PNGase F), 0-glycanase, and Sialidase A. Treatment of S-Ecto-eGFP with N-glycanase (PNGase F) showed increased mobility shift and destabilization of the protein due to the loss of heavy N-glycans (FIG. 20B). However, the treatment of S-Ecto-eGFP with 0-glycanase showed a slightly decreased protein mobility shift, which supports the previous findings that S-protein has only two O-linked core-1 derived glycans in its RBD backbone (FIG. 20B). Conversely, treatment of S-Ecto-eGFP with Sialidase A (which removes sialic acid group) either alone or in combination with either N-glycanase or 0-glycanase or together induced a noticeable change in the migration of S-Ecto-eGFP. This decreased mobility shift may be due to the loss of negatively charged sialic acid groups in the N- and O-linked glycans (FIG. 20B). These results indicate that the SARS-CoV-2 Spike protein has both highly sialylated N- and O-linked glycans.

Binding of the S Ectodomain Constructs to the hACE2 Receptor

The binding of the purified S ectodomains to hACE2 was tested using SPR (FIG. 21) to test their activity relative to wild-type proteins made in human 293T cells. The interaction between hACE2 and wild type SARS-CoV-2 spike trimers has a K_{D} of 14.7 nM.^{9, 21} The interaction between hACE2 and S-Ecto-eGFP, S-Ecto-HexaPro(+F), and S-Ecto-HexaPro(−F) have K_{D} values of 55.1 nM, 72.5 nM, and 20.3 nM, respectively (FIG. 21B). Although the affinity of these purified recombinant proteins to hACE2 is lower (higher K_{D}), they share the same order of magnitude as the wild type SARS-CoV2 spike trimer indicating that they share a high affinity for hACE2. The binding between wild-type SARS-CoV2 RBD and hACE2 has a K_{D} of 4.7 nM.⁹ The binding between hACE2 and the S-RBD-eGFP construct was 185 nM. This construct binds with only moderate affinity compared to the wild-type SARS-CoV2 RBD. The high-affinity binding of the spike ectodomain constructs and the moderate affinity of the S-RBD-eGFP construct demonstrates that the constructs are properly folded and functionally active like native SARS-CoV-2 spike proteins.

Targeting ACE2 Receptor Inhibits Binding by the S-Ecto-eGFP

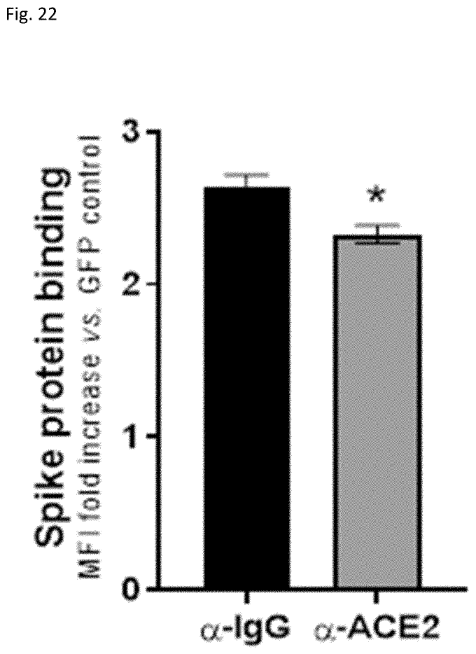

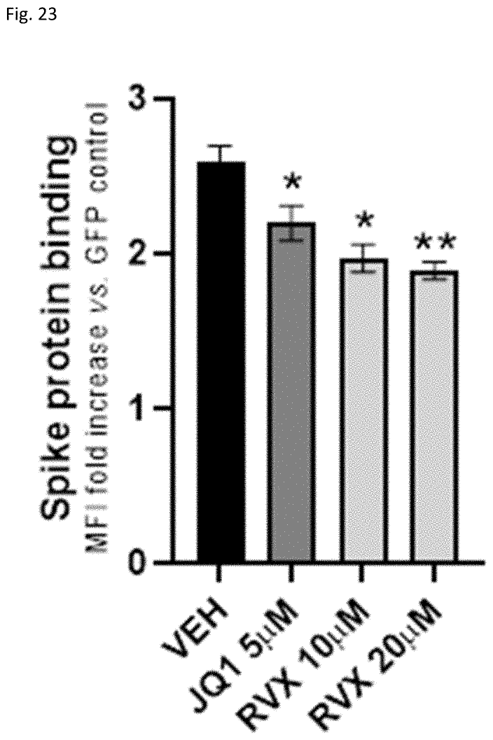

The functional binding of S-Ecto-eGFP to hACE2 on bronchial epithelial Calu-3 cells was additionally evaluated via flow cytometry. Calu-3 cells incubated with S-Ecto-eGFP demonstrated a 2.6-fold greater median GFP fluorescence intensity (MFI) than cells incubated with GFP-His tag control (FIGS. 22, 23). Blocking the hACE2 receptor with anti-ACE2 significantly inhibited S-Ecto-eGFP binding compared to IgG control (FIG. 22).

Two BET inhibitors were used in this study to downregulate hACE2 expression on Calu-3 cells.²⁹ Both JQ1 and apabetalone (RVX-208) significantly inhibited S-Ecto-eGFP binding compared to vehicle control (FIG. 23). JQ1 is a pan-BET inhibitor with equal affinity for the two BET bromodomains (BD1 and BD2), while the clinically advanced RVX-208 preferentially targets the BD2 bromodomain. RVX-208 has an established favorable safety profile in clinical trials for cardiovascular indications and is now the focus of a clinical trial for COVID-19 treatment compared to standard of care (Clinical trial identifier: NCT04894266).^{30, 42, 43} In addition to reducing SARS-CoV-2 infection, RVX-208 may play a pivotal role in controlling hyperinflammatory immune responses that can cause long-term tissue damage in patients.^{28, 30, 42, 44, 45} BET inhibitors have also been reported to decrease the expression of immune inhibitory receptors, such as PD-L1 and LAG3 which hinder T-cell function.⁴⁶ High expression of LAG3 correlates with more severe disease in COVID-19 patients, with normalization of receptor levels witnessed during recovery.⁴⁷

Discussion

We demonstrated a robust and reliable method for purifying multiple constructs of the S ectodomain that is easy to replicate and can be adapted to different variants of the spike domain to obtain purified active proteins. For all constructs tested, we found that the overall yield was higher when Tni cells were used, with the S-Ecto-HexaPro(+F) specifically showing a large 3.7× increase in yield. Compared to the similar construct expressed in mammalian FreeStyle 293-F cells by Hsieh C L and coworkers, we find that the mammalian cell system has a 2.3× increase in yield over our method; however, the ease of culturing insect cells over mammalian cells may prove appealing.³⁵ Our research also shows the importance of the six stabilizing proline mutations in insect cells, as the yield from Tni cells of S-Ecto-HexaPro(−F) and S-Ecto-HexaPro(+F) constructs showed a 5× and 22× increase, respectively when compared to S-Ecto-eGFP. It is, however, possible that the change in yield is due to the additional eGFP being produced. These results closely match what was seen by Hsieh et al. (2020), where spike ectodomain with the same HexaPro as our constructs was seen to have ten-fold higher expression as a two proline mutant, and the two proline mutant was developed to have higher and more stable expression than the native spike ectodomain.^{21, 35} When our results are compared to other published methods for producing full length spike ectodomain, we find that Wrap et al. (2020) using FreeStyle 293 cells had a yield of 0.5 mg/L, while Hsieh et al. (2020) using ExpiCHO cells had a yield of 32.5 mg/L.^{21, 35} We find our yields closest to Wrap et al. (2020), with lower yields using S-Ecto-eGFP (0.16 mg/L from SF9 cells and 0.2 mg/L from Tni cells) and higher yields with S-Ecto-HexPro(+F) (1.2 mg/L with Sf9 cells and 4.4 mg/L with Tni cells) and S-Ecto-HexPro(−F) (0.58 mg/L with Sf9 cells and 1.0 mg/L with Tni cells). The yield for all of our constructs was lower than what was reported by Hsieh et al. (2020). When we compare our S-RBD-eGFP construct to our previously published RBD with maltose binding protein tag (RBD-MBP) expressed in E. coli BL21(DE3) cells using CyDisCO to form the required disulfide bonds, we find that our method yields 3.5 mg/L using Sf9 cells opposed to the 0.5 mg/L using the E. coli system, and the S-RBD-eGFP construct is likely glycosylated.⁴⁸

As new SARS-nCoV-2 variants continue to emerge, future studies adding to the body of research about the novel coronavirus as well as the development of countermeasures against the COVID-19 disease itself will be required. The process demonstrated provides an entry for small laboratories to begin research into SARS-CoV-2 S protein in vitro or in vivo studies, as well as a rapid start-up for laboratories that are already working in the field.

We also demonstrated that the novel S-Ecto-eGFP construct could be used to measure changes in functional SARS-CoV-2 spike protein binding to commonly infected cells, such as those of the lung epithelium. S1-Ecto-eGFP binding, quantified by flow cytometry, is significantly decreased by blocking the ACE2 receptor with ACE2 antibodies or by reducing ACE2 expression with BET inhibitors. This is relevant because S-Ecto-eGFP functionality can be monitored through ACE2 receptor binding, and we see a decrease in binding as a dose-dependent response to these inhibitors.

Further, the S-Ecto-eGFP construct has already successfully been used in a published study concerning the effect of bromelain on lessening the interaction between the S-Ecto-eGFP and VeroE6 cells and the diminished levels of SARS-CoV-2 infection in VeroE6 cells.³⁹ In that study, the S-Ecto-eGFP construct was used for multiple experiments that included assessing ACE2 receptor binding, a serological assay where the S-Ecto-eGFP is recognized by COVID-19 positive patient samples, and treatment with bromelain testing for susceptibility to cleavage by the protease.

### Example 5: Insect Cell Expression of Variant SARS-CoV-2 Peptides

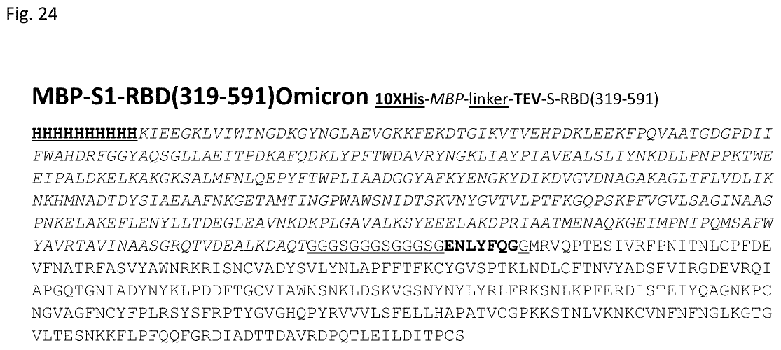

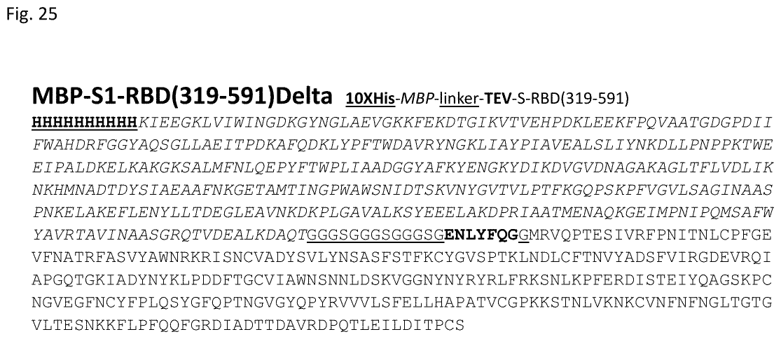

Natural variants of the original SARS-CoV-2 pathogen have been discovered during the course of the ongoing COVID-19 pandemic. Such natural variants have mutations in key viral proteins, including the surface glycoprotein (S protein). Accordingly, the inventors have produced sequences encoding the S proteins derived from “variants of concern”, i.e., variants which pose an increased risk to public health, to produce the S proteins in insect cells, according to the methods of the instant disclosure. Therefore, disclosed herein are further recombinant proteins shown in FIGS. 24 and 25 comprising SEQ ID NOs: 30 and 31. The variant recombinant S proteins are derived from the “omicron” and “delta” variants of concern, respectively.

It is anticipated that the variant recombinant S proteins will function similarly to the other recombinant proteins disclosed herein. However, the omicron and delta variant proteins are expected to induce an immune response tailored to the corresponding variant of concern from which the sequence is derived. Similarly, it is anticipated that the recombinant proteins comprising omicron or delta variant of concern S protein, or a fragment thereof, e.g., SEQ ID NOs: 30 and 31, will be bound by antibodies specific for the variant of concern from which the S protein is derived. Thus, by way of example, but not by way of limitation, it is anticipated that a recombinant protein of the instant disclosure which comprises omicron S protein, or a fragment thereof, e.g., the RBD or the ectodomain, will bind to antibodies directed to SARS-CoV-2 omicron variant, and may be used in methods of the instant disclosure to detect such antibodies, and, thus, exposure of an individual to SARS-CoV-2, or a SARS-CoV-2 variant of concern.

### REFERENCES

Hamre, D. and J. J. Procknow, 1966, A new virus isolated from the human respiratory tract. Proc Soc Exp Biol Med. 121(1): p. 190-3.
2. Bonilla-Aldana, D. K., Y. Holguin-Rivera, I. Cortes-Bonilla, M. C. Cardona-Trujillo, A. Garcia-Barco, H. A. Bedoya-Arias, A. A. Rabaan, R. Sah, and A. J. Rodriguez-Morales, 2020, Coronavirus infections reported by ProMED, February 2000-January 2020. Travel Med Infect Dis. 35: p. 101575.
3. Skariyachan, S., S. B. Challapilli, S. Packirisamy, S. T. Kumargowda, and V. S. Sridhar, 2019, Recent Aspects on the Pathogenesis Mechanism, Animal Models and Novel Therapeutic Interventions for Middle East Respiratory Syndrome Coronavirus Infections. Front Microbiol. 10: p. 569.
4. Walls, A. C., Y. J. Park, M. A. Tortorici, A. Wall, A. T. McGuire, and D. Veesler, 2020, Structure, Function, and Antigenicity of the SARS-CoV-2 Spike Glycoprotein. Cell. 183(6): p. 1735.
5. Chen, Y., Q. Liu, and D. Guo, 2020, Emerging coronaviruses: Genome structure, replication, and pathogenesis. J Med Virol. 92(10): p. 2249.
6. Paules, C. I., H. D. Marston, and A. S. Fauci, 2020, Coronavirus Infections-More Than Just the Common Cold. JAMA. 323(8): p. 707-708.
7. Ghinai, I., T. D. McPherson, J. C. Hunter, H. L. Kirking, D. Christiansen, K. Joshi, R. Rubin, S. Morales-Estrada, S. R. Black, M. Pacilli, M. J. Fricchione, R. K. Chugh, K. A. Walblay, N. S. Ahmed, W. C. Stoecker, N. F. Hasan, D. P. Burdsall, H. E. Reese, M. Wallace, C. Wang, D. Moeller, J. Korpics, S. A. Novosad, I. Benowitz, M. W. Jacobs, V. S. Dasari, M. T. Patel, J. Kauerauf, E. M. Charles, N. O. Ezike, V. Chu, C. M. Midgley, M. A. Rolfes, S. I. Gerber, X. Lu, S. Lindstrom, J. R. Verani, J. E. Layden, and C.-I.T. Illinois, 2020, First known person-to-person transmission of severe acute respiratory syndrome coronavirus 2 (SARS-CoV-2) in the USA. Lancet. 395(10230): p. 1137-1144.
8. Hu, B., H. Guo, P. Zhou, and Z. L. Shi, 2021, Characteristics of SARS-CoV-2 and COVID-19. Nat Rev Microbiol. 19(3): p. 141-154.
9. Lan, J., J. Ge, J. Yu, S. Shan, H. Zhou, S. Fan, Q. Zhang, X. Shi, Q. Wang, L. Zhang, and X. Wang, 2020, Structure of the SARS-CoV-2 spike receptor-binding domain bound to the ACE2 receptor. Nature. 581(7807): p. 215-220.
10. World Health Organization, W. WHO Director-General's Opening Remarks at the Media Briefing on COVID-19—11 Mar. 2020. [Web Page] 2020 [cited 2021 13 Jul. 2021]; Available from: https://www.who.int/director-general/speeches/detail/who-director-general-s-opening-remarks-at-the-media-briefing-on-covid-19—-11-march-2020.
11. Soliman, M. S., M. AbdelFattah, S. M. N. Aman, L. M. Ibrahim, and R. K. Aziz, 2021, A Gapless, Unambiguous RNA Metagenome-Assembled Genome Sequence of a Unique SARS-CoV-2 Variant Encoding Spike S8131 and ORF1a A859V Substitutions. OMICS. 25(2): p. 123-128.
12. Saif, R., T. Mahmood, A. Ejaz, S. Zia, and A. R. Qureshi, 2021, Whole genome comparison of Pakistani Corona virus with Chinese and US Strains along with its predictive severity of COVID-19. Gene Rep. 23: p. 101139.
13. Giovanetti, M., F. Benedetti, G. Campisi, A. Ciccozzi, S. Fabris, G. Ceccarelli, V.
Tambone, A. Caruso, S. Angeletti, D. Zella, and M. Ciccozzi, 2021, Evolution patterns of SARS-CoV-2: Snapshot on its genome variants. Biochem Biophys Res Commun. 538: p. 88-91.
14. Lau, S. Y., P. Wang, B. W. Mok, A. J. Zhang, H. Chu, A. C. Lee, S. Deng, P. Chen, K. H.
Chan, W. Song, Z. Chen, K. K. To, J. F. Chan, K. Y. Yuen, and H. Chen, 2020, Attenuated SARS-CoV-2 variants with deletions at the S1/S2 junction. Emerg Microbes Infect. 9(1): p. 837-842.
15. Korber, B., W. M. Fischer, S. Gnanakaran, H. Yoon, J. Theiler, W. Abfalterer, N. Hengartner, E. E. Giorgi, T. Bhattacharya, B. Foley, K. M. Hastie, M. D. Parker, D. G. Partridge, C. M. Evans, T. M. Freeman, T. I. de Silva, C.-G. G. Sheffield, C. McDanal, L. G. Perez, H. Tang, A. Moon-Walker, S. P. Whelan, C. C. LaBranche, E. O. Saphire, and D. C. Montefiori, 2020, Tracking Changes in SARS-CoV-2 Spike: Evidence that D614G Increases Infectivity of the COVID-19 Virus. Cell. 182(4): p. 812-827 e19.
16. Shiehzadegan, S., N. Alaghemand, M. Fox, and V. Venketaraman, 2021, Analysis of the Delta Variant B.1.617.2 COVID-19. Clin Pract. 11(4): p. 778-784.
17. Cele, S., L. Jackson, D. S. Khoury, K. Khan, T. Moyo-Gwete, H. Tegally, J. E. San, D. Cromer, C. Scheepers, D. G. Amoako, F. Karim, M. Bernstein, G. Lustig, D. Archary, M. Smith, Y. Ganga, Z. Jule, K. Reedoy, S. H. Hwa, J. Giandhari, J. M. Blackburn, B. I. Gosnell, S. S. Abdool Karim, W. Hanekom, S. A. Ngs, C.-K. Team, A. von Gottberg, J. N. Bhiman, R. J. Lessells, M. S. Moosa, M. P. Davenport, T. de Oliveira, P. L. Moore, and A. Sigal, 2021, Omicron extensively but incompletely escapes Pfizer BNT162b2 neutralization. Nature.
18. Ferre, V. M., N. Peiffer-Smadja, B. Visseaux, D. Descamps, J. Ghosn, and C. Charpentier, 2021, Omicron SARS-CoV-2 variant: What we know and what we don't. Anaesth Crit Care Pain Med: p. 100998.
19. Garcia-Beltran, W. F., K. J. St Denis, A. Hoelzemer, E. C. Lam, A. D. Nitido, M. L. Sheehan, C. Berrios, O. Ofoman, C. C. Chang, B. M. Hauser, J. Feldman, A. L. Roederer, D. J. Gregory, M. C. Poznansky, A. G. Schmidt, A. J. Iafrate, V. Naranbhai, and A. B. Balazs, 2022, mRNA-based COVID-19 vaccine boosters induce neutralizing immunity against SARS-CoV-2 Omicron variant. Cell.
20. Tai, W., L. He, X. Zhang, J. Pu, D. Voronin, S. Jiang, Y. Zhou, and L. Du, 2020, Characterization of the receptor-binding domain (RBD) of 2019 novel coronavirus: implication for development of RBD protein as a viral attachment inhibitor and vaccine. Cell Mol Immunol. 17(6): p. 613-620.
21. Wrapp, D., N. Wang, K. S. Corbett, J. A. Goldsmith, C. L. Hsieh, O. Abiona, B. S. Graham, and J. S. McLellan, 2020, Cryo-E111 structure of the 2019-nCoV spike in the prefusion conformation. Science. 367(6483): p. 1260-1263.
22. Wrapp, D., N. Wang, K. S. Corbett, J. A. Goldsmith, C. L. Hsieh, O. Abiona, B. S. Graham, and J. S. McLellan, 2020, Cryo EM Structure of the 2019-nCoV Spike in the Prefusion Conformation. bioRxiv.
23. Huang, Y., C. Yang, X. F. Xu, W. Xu, and S. W. Liu, 2020, Structural and functional properties of SARS-CoV-2 spike protein: potential antivirus drug development for COVID-19. Acta Pharmacol Sin. 41(9): p. 1141-1149.
24. Duan, L., Q. Zheng, H. Zhang, Y. Niu, Y. Lou, and H. Wang, 2020, The SARS-CoV-2 Spike Glycoprotein Biosynthesis, Structure, Function, and Antigenicity: Implications for the Design of Spike-Based Vaccine Immunogens. Front Immunol. 11: p. 576622.
25. Watanabe, Y., J. D. Allen, D. Wrapp, J. S. McLellan, and M. Crispin, 2020, Site-specific glycan analysis of the SARS-CoV-2 spike. Science. 369(6501): p. 330-333.
26. Watanabe, Y., J. D. Allen, D. Wrapp, J. S. McLellan, and M. Crispin, 2020, Site-specific analysis of the SARS-CoV-2 glycan shield. bioRxiv.
27. Qiao, Y., X. M. Wang, R. Mannan, S. Pitchiaya, Y. Zhang, J. W. Wotring, L. Xiao, D. R. Robinson, Y. M. Wu, J. C. Tien, X. Cao, S. A. Simko, I. J. Apel, P. Bawa, S. Kregel, S. P. Narayanan, G. Raskind, S. J. Ellison, A. Parolia, S. Zelenka-Wang, L. McMurry, F. Su, R. Wang, Y. Cheng, A. D. Delekta, Z. Mei, C. D. Pretto, S. Wang, R. Mehra, J. Z. Sexton, and A. M. Chinnaiyan, 2020, Targeting transcriptional regulation of SARS-CoV-2 entry factors ACE2 and TMPRSS2. Proc Natl Acad Sci USA.
28. Lara-Urena, N. and M. Garcia-Dominguez, 2021, Relevance of BET Family Proteins in SARS-CoV-2 Infection. Biomolecules. 11(8).
29. Gilham, D., A. L. Smith, L. Fu, D. Y. Moore, A. Muralidharan, S. P. M. Reid, S. C. Stotz, J. O. Johansson, M. Sweeney, N. C. W. Wong, E. Kulikowski, and D. El-Gamal, 2021, Bromodomain and Extraterminal Protein Inhibitor, Apabetalone (RVX-208), Reduces ACE2 Expression and Attenuates SARS-Cov-2 Infection In Vitro. Biomedicines. 9(4).
30. Mills, R. J., S. J. Humphrey, P. R. J. Fortuna, M. Lor, S. R. Foster, G. A. Quaife-Ryan, R. L. Johnston, T. Dumenil, C. Bishop, R. Rudraraju, D. J. Rawle, T. Le, W. Zhao, L. Lee, C. Mackenzie-Kludas, N. R. Mehdiabadi, C. Halliday, D. Gilham, L. Fu, S. J. Nicholls, J. Johansson, M. Sweeney, N. C. W. Wong, E. Kulikowski, K. A. Sokolowski, B. W. C. Tse, L. Devilee, H. K. Voges, L. T. Reynolds, S. Krumeich, E. Mathieson, D. Abu-Bonsrah, K. Karavendzas, B. Griffen, D. Titmarsh, D. A. Elliott, J. McMahon, A. Suhrbier, K. Subbarao, E. R. Porrello, M. J. Smyth, C. R. Engwerda, K. P. A. MacDonald, T. Bald, D. E. James, and J. E. Hudson, 2021, BET inhibition blocks inflammation-induced cardiac dysfunction and SARS-CoV-2 infection. Cell. 184(8): p. 2167-2182 e22.
31. Ikonomou, L., Y. J. Schneider, and S. N. Agathos, 2003, Insect cell culture for industrial production of recombinant proteins. Appl Microbiol Biotechnol. 62(1): p. 1-20.
32. Jarvis, D. L., 2009, Baculovirus-insect cell expression systems. Methods Enzymol. 463: p. 191-222.
33. Waldo, G. S., B. M. Standish, J. Berendzen, and T. C. Terwilliger, 1999, Rapid protein-folding assay using green fluorescent protein. Nat Biotechnol. 17(7): p. 691-5.
34. Poppenborg, L., K. Friehs, and E. Flaschel, 1997, The green fluorescent protein is a versatile reporter for bioprocess monitoring. J Biotechnol. 58(2): p. 79-88.
35. Hsieh, C. L., J. A. Goldsmith, J. M. Schaub, A. M. DiVenere, H. C. Kuo, K. Javanmardi, K. C. Le, D. Wrapp, A. G. Lee, Y. Liu, C. W. Chou, P. O. Byrne, C. K. Hjorth, N. V. Johnson, J. Ludes-Meyers, A. W. Nguyen, J. Park, N. Wang, D. Amengor, J. J. Lavinder, G. C. Ippolito, J. A. Maynard, I. J. Finkelstein, and J. S. McLellan, 2020, Structure-based design of prefusion-stabilized SARS-CoV-2 spikes. Science. 369(6510): p. 1501-1505.
36. Meier, S., S. Guthe, T. Kiefhaber, and S. Grzesiek, 2004, Foldon, the natural trimerization domain of T4 fibritin, dissociates into a monomeric A-state form containing a stable beta-hairpin: atomic details of trimer dissociation and local beta-hairpin stability from residual dipolar couplings. J Mol Biol. 344(4): p. 1051-69.
37. Pallesen, J., N. Wang, K. S. Corbett, D. Wrapp, R. N. Kirchdoerfer, H. L. Turner, C. A. Cottrell, M. M. Becker, L. Wang, W. Shi, W. P. Kong, E. L. Andres, A. N. Kettenbach, M. R. Denison, J. D. Chappell, B. S. Graham, A. B. Ward, and J. S. McLellan, 2017, Immunogenicity and structures of a rationally designed prefusion MERS-CoV spike antigen. Proc Natl Acad Sci USA. 114(35): p. E7348-E7357.
38. Edwards, R. J., K. Mansouri, V. Stalls, K. Manne, B. Watts, R. Parks, K. Janowska, S. M. C. Gobeil, M. Kopp, D. Li, X. Lu, Z. Mu, M. Deyton, T. H. Oguin, J. Sprenz, W. Williams, K. Saunders, D. Montefiori, G. D. Sempowski, R. Henderson, M. Alam, B. F. Haynes, and P. Acharya, 2020, Cold sensitivity of the SARS-CoV-2 spike ectodomain. bioRxiv.
39. Sagar, S., A. K. Rathinavel, W. E. Lutz, L. R. Struble, S. Khurana, A. T. Schnaubelt, N. K. Mishra, C. Guda, N. Y. Palermo, M. J. Broadhurst, T. Hoffmann, K. W. Bayles, S. P. M. Reid, G. E. O. Borgstahl, and P. Radhakrishnan, 2021, Bromelain inhibits SARS-CoV-2 infection via targeting ACE-2, TMPRSS2, and spike protein. Clin Transl Med. 11(2): p. e281.
40. Shajahan, A., N. T. Supekar, A. S. Gleinich, and P. Azadi, 2020, Deducing the N-and O-glycosylation profile of the spike protein of novel coronavirus SARS-CoV-2. Glycobiology. 30(12): p. 981-988.
41. Zhao, P., J. L. Praissman, O. C. Grant, Y. Cai, T. Xiao, K. E. Rosenbalm, K. Aoki, B. P. Kellman, R. Bridger, D. H. Barouch, M. A. Brindley, N. E. Lewis, M. Tiemeyer, B. Chen, R. J. Woods, and L. Wells, 2020, Virus Receptor Interactions of Glycosylated SARS-CoV-2 Spike and Human ACE2 Receptor. bioRxiv.
42. Tsujikawa, L. M., L. Fu, S. Das, C. Halliday, B. D. Rakai, S. C. Stotz, C. D. Sarsons, D. Gilham, E. Daze, S. Wasiak, D. Studer, K. D. Rinker, M. Sweeney, J. O. Johansson, N. C. W. Wong, and E. Kulikowski, 2019, Apabetalone (RVX-208) reduces vascular inflammation in vitro and in CVD patients by a BET-dependent epigenetic mechanism. Clin Epigenetics. 11(1): p. 102.
43. An Open-Label Study of Apabetalone in Covid Infection [NCT04894266]. [Web Page] 2021 May 20, 2021 [cited 2021 09-02-2021]; Available from: https://clinicaltrials.gov/ct2/show/NCT04894266?term=Apabetalone& draw=2&rank=3
44. Wasiak, S., D. Gilham, E. Daze, L. M. Tsujikawa, C. Halliday, S. C. Stotz, B. D. Rakai, L. Fu, R. Jahagirdar, M. Sweeney, J. O. Johansson, N. C. W. Wong, and E. Kulikowski, 2020, Epigenetic Modulation by Apabetalone Counters Cytokine-Driven Acute Phase Response In Vitro, in Mice and in Patients with Cardiovascular Disease. Cardiovasc Ther. 2020: p. 9397109.
45. Wasiak, S., K. E. Dzobo, B. D. Rakai, Y. Kaiser, M. Versloot, M. Bahj at, S. C. Stotz, L. Fu, M. Sweeney, J. O. Johansson, N. C. W. Wong, E. S. G. Stroes, J. Kroon, and E. Kulikowski, 2020, BET protein inhibitor apabetalone (RVX-208) suppresses pro-inflammatory hyper-activation of monocytes from patients with cardiovascular disease and type 2 diabetes. Clin Epigenetics. 12(1): p. 166.
46. Ozer, H. G., D. El-Gamal, B. Powell, Z. A. Hing, J. S. Blachly, B. Harrington, S. Mitchell, N. R. Grieselhuber, K. Williams, T. H. Lai, L. Alinari, R. A. Baiocchi, L. Brinton, E. Baskin, M. Cannon, L. Beaver, V. M. Goettl, D. M. Lucas, J. A. Woyach, D. Sampath, A. M. Lehman, L. Yu, J. Zhang, Y. Ma, Y. Zhang, W. Spevak, S. Shi, P. Severson, R. Shellooe, H. Carias, G. Tsang, K. Dong, T. Ewing, A. Marimuthu, C. Tantoy, J. Walters, L. Sanftner, H. Rezaei, M. Nespi, B. Matusow, G. Habets, P. Ibrahim, C. Zhang, E. A. Mathe, G. Bollag, J. C. Byrd, and R. Lapalombella, 2018, BRD4 Profiling Identifies Critical Chronic Lymphocytic Leukemia Oncogenic Circuits and Reveals Sensitivity to PLX51107, a Novel Structurally Distinct BET Inhibitor. Cancer Discov. 8(4): p. 458-477.
47. Herrmann, M., S. Schulte, N. H. Wildner, M. Wittner, T. T. Brehm, M. Ramharter, R. Woost, A. W. Lohse, T. Jacobs, and J. Schulze Zur Wiesch, 2020, Analysis of Co-inhibitory Receptor Expression in COVID-19 Infection Compared to Acute Plasmodium falciparum Malaria: LAG-3 and TIM-3 Correlate With T Cell Activation and Course of Disease. Front Immunol. 11: p. 1870.
48. Prahlad, J., L. R. Struble, W. E. Lutz, S. A. Wallin, S. Khurana, A. Schnaubelt, M. J. Broadhurst, K. W. Bayles, and G. E. O. Borgstahl, 2021, CyDisCo production of functional recombinant SARS-CoV-2 spike receptor binding domain. Protein Sci. 30(9): p. 1983-1990.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A recombinant protein comprising: (i) a SARS-CoV-2 polypeptide sequence derived from the spike (“S”) protein or a variant thereof, and (ii) one or more heterologous polypeptide sequences selected from a purification tag, a detectable label, a flexible linker, a cleavage site to allow for tag removal after purification, and a secretion signal peptide.

2. The recombinant protein of claim 1, wherein the furin site “RRAR” in the polypeptide is genetically engineered so as not to be cleaved by furin, optionally wherein the furin site is engineered to “GSAS.”

3. The recombinant protein of claim 1, wherein the SARS-CoV-2 polypeptide sequence comprises a fragment of the S protein including amino acids 319-591, or a variant thereof.

4. The recombinant protein of claim 1, wherein the detectable label comprises green fluorescent protein (GFP) or enhanced green fluorescent protein (eGFP).

5. The recombinant protein of claim 1, comprising one or more mutations selected from the group consisting of: F817P, A892P, A899P, A942P, K986P, and V987P, relative to SEQ ID NO: 14.

6. The recombinant protein of claim 1, wherein the flexible linker comprises GGGGSGGGGSGG (SEQ ID NO: 34).

7. The recombinant protein of claim 1, wherein the cleavage site to allow for tag removal after purification comprises a Tobacco Etch Virus nuclear-inclusion-a endopeptidase (TEV protease) recognition sequence: GENLYFQG (SEQ ID NO: 35).

8. The recombinant protein of claim 1, wherein the secretion signal peptide comprises MFLLTTKRT (SEQ ID NO: 36).

9. The recombinant protein of claim 1, further comprising a foldon trimerization domain.

10. The recombinant protein of claim 1, comprising a solubility enhancer peptide comprising maltose binding protein (MBP).

11. The recombinant protein of claim 10, wherein the maltose binding protein (MBP) comprises a GGSK₁₀ sequence (SEQ ID NO: 38) at its N terminus or C terminus.

12. The recombinant protein of claim 1, wherein the heterologous polypeptide sequence comprises (a) a purification tag comprising a HIS tag; (b) a detectable label comprising Green Fluorescent Protein or enhanced Green Fluorescent Protein; (c) a flexible linker comprising GGGGSGGGGSGG (SEQ ID NO: 34); (d) a cleavage site to allow for tag removal after purification comprising a Tobacco Etch Virus nuclear-inclusion-a endopeptidase (TEV protease) recognition sequence, GENLYFQG (SEQ ID NO: 35); (e) a secretion signal peptide comprising MFLLTTKRT (SEQ ID NO: 36); (f) a foldon trimerization domain; wherein the “S” protein or fragment thereof comprises the mutations F817P, A892P, A899P, A942P, K986P, and V987P, relative to SEQ ID NO: 14.

13. The recombinant protein of claim 1, wherein the recombinant protein comprises a sequence selected from the group consisting of SEQ ID NOs: 7-13, 19-25, and 30-31.

14. A pharmaceutical composition comprising the recombinant protein of claim 1.

15. The pharmaceutical composition of claim 14, further comprising a pharmaceutically acceptable carrier.

16. The pharmaceutical composition of claim 15, further comprising an adjuvant.

17. A method of inducing an immune response against SARS-CoV-2 in a subject in need thereof, the method comprising administering to the subject an effective amount of the pharmaceutical composition of claim 14.

18. The method of claim 17, wherein the immune response against SARS-CoV-2 in the subject comprises a cellular immune response, a humoral immune response, or both a cellular and a humoral immune response.

19. A method for identifying whether a subject has been exposed to SARS-CoV-2, the method comprising:
(a) obtaining a sample from the subject;
(b) contacting the sample with the recombinant protein of claim 1 under conditions that allow SARS-CoV-2 antibodies, if present in the sample, to bind to the recombinant protein and form an antibody-antigen complex; and
(c) detecting the complex.

20. The method of claim 19, wherein the complex is detected by contacting the complex with a secondary antibody that binds the complex and comprises a detectable label, optionally wherein the secondary antibody is an anti-human antibody that binds human SARS-CoV-2 antibodies and comprises a fluorometric label or colorimetric label.
